(19) United States
(12) Patent Application Publication
(10) Pub. No.: US 2026/0123872 A1
(43) Pub. Date: May 7, 2026

(54) Electrocardiogram-Based Deep Learning for Hypertension Prediction

(71) Applicants: The Broad Institute, Inc., Cambridge, MA (US); The General Hospital Corporation, Boston, MA (US)

(72) Inventors: Patrick T. Ellinor, Boston, MA (US); Mostafa Al-Alusi, Cambridge, MA (US); Samuel F Friedman, Cambridge, MA (US)

(73) Assignees: The Broad Institute, Inc., Cambridge, MA (US); The General Hospital Corporation, Boston, MA (US)

(21) Appl. No.: 19/379,537

(22) Filed: Nov. 4, 2025

Related U.S. Application Data
(60) Provisional application No. 63/716,507, filed on Nov. 5, 2024.

(51) Int. Cl. A61B 5/346; A61B 5/00

(52) U.S. Cl. CPC A61B 5/346; A61B 5/7267

(57) ABSTRACT

Electrocardiogram-based deep learning for hypertension prediction is described. An electrocardiogram analysis module may include a data preprocessor configured to normalize an electrocardiogram to generate a standardized input for electrocardiogram-based hypertension prediction. The electrocardiogram analysis module may further include a deep learning model including a neural network trained to identify features associated with hypertension from the standardized input and at least one dense layer trained to generate a hypertension risk prediction based on the identified features. The hypertension risk prediction may comprise a probability score indicating a likelihood of hypertension.

### RELATED APPLICATION

This application claims priority to U.S. Provisional Patent Application Ser. No. 63/716,507, filed Nov. 5, 2024, entitled “Electrocardiogram-Based Deep Learning for Hypertension Prediction,” the entire disclosure of which is hereby incorporated by reference herein in its entirety.

### STATEMENT REGARDING GOVERNMENT SUPPORT

This invention was made with government support under Grant Nos. HL092577 and HL157635 awarded by the National Institutes of Health. The government has certain rights in the invention.

### BACKGROUND

Hypertension, also known as high blood pressure, is a medical condition characterized by persistently elevated pressure within blood vessels. Hypertension is a risk factor for cardiovascular diseases, including heart disease, as well as stroke and kidney damage. Hypertension affects millions of people worldwide and is often asymptomatic.

Current methods for diagnosing and monitoring hypertension typically rely on periodic blood pressure measurements taken in clinical settings. These measurements may not always accurately reflect a patient's true blood pressure due to factors such as anxiety-induced hypertension or masked hypertension. Additionally, the intermittent nature of these measurements may fail to capture variations in blood pressure that occur throughout the day.

Electrocardiography is a widely available, non-invasive diagnostic tool used in various healthcare settings. Traditionally, electrocardiogram (ECG) interpretation has focused on identifying specific cardiac abnormalities or arrhythmias. However, the potential of the ECG to provide comprehensive cardiovascular risk assessment, particularly for hypertension and its associated complications, has not been realized in current clinical practice.

### SUMMARY

Electrocardiogram-based deep learning for hypertension prediction is described. An electrocardiogram analysis module may include a data preprocessor configured to normalize an electrocardiogram to generate a standardized input for electrocardiogram-based hypertension prediction. The electrocardiogram analysis module may further include a deep learning model including a neural network trained to identify features associated with hypertension from the standardized input and at least one dense layer trained to generate a hypertension risk prediction based on the identified features. The hypertension risk prediction may comprise a probability score indicating a likelihood of hypertension.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example implementation that is operable to employ electrocardiogram-based deep learning for hypertension prediction as described herein.

FIG. 2 depicts an example training process that may be used in generating the at least one deep learning model of the electrocardiogram (ECG) analysis module of FIG. 1.

FIG. 3 illustrates an example implementation of a convolutional block structure that may be used in the convolutional layers of the convolutional neural network (CNN) of FIG. 2.

FIG. 4 depicts an example implementation of using the at least one deep learning model to assess electrocardiogram data for a hypertension prediction.

FIG. 5 depicts an example procedure for training and validating a machine learning model to output a hypertension prediction according to one or more implementations.

FIG. 6 depicts an example procedure for generating a hypertension prediction from electrocardiogram data using a machine learning model according to one or more implementations.

FIG. 7 illustrates an example system including various components of an example device that can be implemented as any type of computing device as described and/or utilized with reference to FIGS. 1-6 to implement the techniques described herein.

FIGS. 8A and 8B illustrate a summary of a study overview for the electrocardiogram-based deep learning for hypertension prediction.

FIG. 9 depicts an analysis comparing hazard ratios for various cardiovascular outcomes between baseline hypertension and high HTN-AI risk.

FIG. 10 depicts an analysis of model performance in the internal and external test sets for an electrocardiogram-based deep learning model for hypertension prediction.

FIG. 11 depicts an analysis of cumulative incidence of hypertension over time based on hypertension risk prediction.

FIG. 12 depicts a set of graphs showing cumulative incidence of different cardiovascular outcomes over time, as stratified by HTN-AI risk.

FIG. 13 depicts an analysis of a cumulative incidence of all-cause mortality, as stratified by HTN-AI risk and baseline hypertension status and by quintile of HTN-AI score.

FIG. 14 depicts an analysis of a cumulative incidence of heart failure, as stratified by HTN-AI risk and baseline hypertension status and by quintile of HTN-AI score.

FIG. 15 depicts an analysis of a cumulative incidence of myocardial infarction, as stratified by HTN-AI risk and baseline hypertension status and by quintile of HTN-AI score.

FIG. 16 depicts an analysis of a cumulative incidence of stroke, as stratified by HTN-AI risk and baseline hypertension status and by quintile of HTN-AI score.

FIG. 17 depicts an analysis of a cumulative incidence of aortic dissection or rupture, as stratified by HTN-AI risk and baseline hypertension status and by quintile of HTN-AI score.

FIG. 18 depicts an analysis comparing hazard ratios for various cardiovascular outcomes between HTN-AI and baseline blood pressure.

FIG. 19 depicts an analysis comparing hazard ratios for associations between HTN-AI score and various cardiovascular outcomes in patients with normal ECGs.

### DETAILED DESCRIPTION

### Overview

As mentioned above, hypertension is a risk factor for cardiovascular diseases, including heart disease, stroke, and kidney damage. As used herein, “hypertension” may be defined as a medical condition characterized by persistently elevated pressure within blood vessels. Hypertension may be indicated by a baseline systolic blood pressure measurement of at least 140 mmHg, or a baseline diastolic blood pressure measurement of at least 90 mmHg. The specific thresholds and criteria for diagnosing hypertension may vary based on clinical guidelines and/or patient characteristics. Typically, hypertension is diagnosed in response to multiple elevated measurements over time rather than a single elevated measurement.

Early detection and risk stratification of hypertension may be beneficial for implementing timely interventions and potentially improving patient outcomes. However, diagnosing hypertension can be challenging due to its often asymptomatic nature and the variability of blood pressure measurements. Hypertension may not present with clear symptoms, making it difficult for patients to recognize the condition without medical evaluation. Additionally, hypertension may develop gradually, making early detection challenging without regular monitoring. The variability of blood pressure readings, influenced by factors such as stress, time of day, and recent activities, may further complicate the diagnostic process. Moreover, the reliance on office-based blood pressure measurements may miss cases of masked hypertension, where blood pressure is normal in clinical settings but elevated in daily life, potentially leading to underdiagnosis and inadequate treatment.

Electrocardiography is a widely available, non-invasive, and relatively inexpensive diagnostic tool used in various healthcare settings. The electrocardiogram (ECG) records the electrical activity of the heart over time, typically using 12 leads placed on the body surface. These leads capture electrical signals from different angles, providing a comprehensive view of the electrical activity of the heart. However, the potential of the ECG to provide comprehensive cardiovascular risk assessment, particularly for hypertension and its associated complications, has not been realized in current clinical practice.

To overcome these issues, electrocardiogram-based deep learning for hypertension prediction is disclosed herein. In accordance with the described techniques, a deep learning model is used to process ECG data and generate a hypertension risk prediction. The deep learning model is trained on vast quantities of ECG data in order to identify subtle patterns in the ECG waveform that may indicate current hypertension and/or an increased risk of future hypertension. Moreover, the techniques described herein provide a model architecture that may be adapted to output one or more auxiliary predictions, non-limiting examples of which include an age prediction, a sex prediction, and a prediction of antihypertensive medication use.

By way of example, the techniques described herein enable generation of a machine learning model (e.g., a deep learning model) that is able to output an accurate hypertension risk prediction from a single ECG. By leveraging the full ECG waveform data, including information that may not be typically used or interpreted during manual clinical analysis, the machine learning model “learns” how to interpret latent information from ECGs that may not be readily interpretable by human observers. By way of example, via the training process described herein, the machine learning model may learn to identify complex patterns that go beyond what is measurable by a human observer. For instance, the machine learning model may include a convolutional neural network (CNN) that, through training, learns to extract features of an input ECG that are relevant to a particular prediction task (or tasks) for which the machine learning model is being trained (e.g., hypertension risk prediction). The machine learning model thus learns ECG features associated with hypertension. These features, which may be captured in ECG feature maps output by the CNN, represent a high-dimensional abstraction of the ECG data. By leveraging this information, the machine learning model may learn to make accurate predictions about hypertension risk that may not be evident from visual inspection or manual measurement performed by a human.

As used herein, “features associated with hypertension” may refer to patterns, characteristics, and/or signals within ECG data that correlate with or indicate the presence of hypertension or an increased risk of developing hypertension, including patterns, characteristics, and/or signals within the ECG data that are not interpretable by a human and/or using manual analysis workflows. The features associated with hypertension may include morphological changes in ECG waveforms, temporal patterns in cardiac electrical activity, voltage amplitude variations, interval measurements, and complex multi-dimensional patterns, just to name a few examples. The features associated with hypertension may encompass both explicit measurements (e.g., QRS voltages, PR intervals, QT intervals, P-wave durations, and T-wave morphologies) as well as latent or implicit patterns that are learned and identified by the machine learning model through the analysis of large training datasets. These features may reflect underlying cardiac structural and functional changes that result from chronic elevated blood pressure.

The techniques described herein represent an advance in computer engineering and provide a substantial advancement over existing practices. The data acquired to prepare the machine learning models are technical data relating to ECG recordings. The methods and systems described herein are more consistent, accurate, and efficient than manual/human analysis, which is prone to bias and does not scale to the amount of ECG data that is generated today.

Furthermore, these techniques may enable non-expert providers to obtain clinically relevant information from an ECG. This could be particularly valuable in resource-limited or time-sensitive settings where specialist expertise may not be immediately available. The machine learning models described herein may be used to identify individuals at high risk of hypertension as soon as the ECG is completed, potentially enabling enhanced clinical decision support and more timely interventions. By providing a rapid and accurate method of assessing hypertension risk from ECG data, these techniques may facilitate both ECG interpretation workflows and the use of ECG data to stratify cardiovascular risk. This approach represents an advancement in the technical fields of computer engineering and medical diagnostics, which may improve patient care and outcomes in the management of hypertension and related cardiovascular diseases.

In some aspects, the techniques described herein relate to a system for electrocardiogram-based hypertension prediction, including: an electrocardiogram analysis module implemented in a non-transitory computer-readable storage medium, the electrocardiogram analysis module including: a data preprocessor configured to normalize an electrocardiogram to generate a standardized input for the electrocardiogram-based hypertension prediction; and a deep learning model including a neural network trained to identify features associated with hypertension from the standardized input and further including at least one dense layer trained to generate a hypertension risk prediction based on the identified features.

In some aspects, the techniques described herein relate to a system, wherein the neural network includes a convolutional neural network.

In some aspects, the techniques described herein relate to a system, wherein the convolutional neural network includes multiple convolutional blocks, each convolutional block including a plurality of convolutional layers, and wherein outputs of at least two convolutional layers of the plurality of convolutional layers are combined via a concatenation.

In some aspects, the techniques described herein relate to a system, wherein the hypertension risk prediction includes a hypertension probability score indicating a likelihood of hypertension or risk of developing hypertension.

In some aspects, the techniques described herein relate to a system, wherein the hypertension risk prediction further includes a hypertension risk stratification that categorizes individuals into risk groups based on the hypertension probability score compared to one or more thresholds.

In some aspects, the techniques described herein relate to a system, wherein the at least one dense layer is further configured to generate auxiliary predictions based on the identified features.

In some aspects, the techniques described herein relate to a system, wherein the auxiliary predictions include at least one of an age prediction, a sex prediction, or a medication prediction.

In some aspects, the techniques described herein relate to a system, wherein the data preprocessor is further configured to upsample and zero-pad the electrocardiogram to generate the standardized input for the electrocardiogram-based hypertension prediction.

In some aspects, the techniques described herein relate to a system, wherein the standardized input includes a time-series of voltage measurements for each lead of the electrocardiogram that are sampled at a standardized frequency over a predetermined time period.

In some aspects, the techniques described herein relate to a system, further including a training module configured to: train the deep learning model using a training sample including a first portion of a model derivation subset of electrocardiogram training data, the electrocardiogram training data including electrocardiograms obtained from healthy patients and from patients diagnosed with hypertension; and refine the trained deep learning model using a development sample including a second portion of the model derivation subset of the electrocardiogram training data.

In some aspects, the techniques described herein relate to a system, wherein the training module is further configured to internally validate the trained and refined deep learning model using an internal test sample including a third portion of the model derivation subset of the electrocardiogram training data.

In some aspects, the techniques described herein relate to a system, wherein the training module is further configured to externally validate the deep learning model using an external test subset of the electrocardiogram training data, wherein the model derivation subset and the external test subset include electrocardiogram recordings from different data sources.

In some aspects, the techniques described herein relate to a method for generating a hypertension risk prediction, said method including: generating, by a data preprocessor, a standardized input for an electrocardiogram that is to be processed by a deep learning model trained to output a hypertension risk prediction; extracting, by a neural network of the deep learning model, features of the standardized input; outputting, by the neural network, an electrocardiogram feature map representing a high-dimensional abstraction of the features of the standardized input; and generating, by an output layer of the deep learning model, the hypertension risk prediction based at least in part on the electrocardiogram feature map, wherein the hypertension risk prediction includes at least a hypertension probability score.

In some aspects, the techniques described herein relate to a method, wherein generating the standardized input includes at least one of normalizing, upsampling, or zero-padding the electrocardiogram, and wherein the standardized input includes a time-series of voltage measurements for each lead of the electrocardiogram that are sampled at a predetermined frequency over a predetermined time period.

In some aspects, the techniques described herein relate to a method, wherein the neural network includes a convolutional neural network having a plurality of convolutional blocks, each convolutional block of the plurality of convolutional blocks including at least one convolutional layer and at least one concatenation, and wherein the output layer includes one or more dense layers.

In some aspects, the techniques described herein relate to a method, further including, generating auxiliary predictions based on the electrocardiogram feature map, the auxiliary predictions including at least one of an age prediction, a sex prediction, or a medication prediction.

In some aspects, the techniques described herein relate to a method for hypertension prediction, including: training a deep learning model to output a hypertension risk prediction, the training including: initially training the deep learning model using a training sample of a model derivation subset of electrocardiogram training data by adjusting weights and biases of the deep learning model based on a difference between an output of the deep learning model for the hypertension risk prediction and a ground truth label; and refining the initially trained deep learning model using a development sample of the model derivation subset of the electrocardiogram training data, the refining including adjusting hyperparameters of the deep learning model; and generating the hypertension risk prediction for an individual using the trained deep learning model, the generating including: generating, by a data preprocessor operatively connected to the trained deep learning model, a standardized input of an electrocardiogram obtained from the individual by preprocessing the electrocardiogram; extracting, by the trained deep learning model, features of the standardized input; generating, by the trained deep learning model, an electrocardiogram feature map summarizing the features of the standardized input; and outputting, by the trained deep learning model, the hypertension risk prediction based on the electrocardiogram feature map.

In some aspects, the techniques described herein relate to a method, wherein the training further includes: internally validating the refined deep learning model using an internal test sample of the model derivation subset of the electrocardiogram training data; and externally validating the internally validated deep learning model using an external test subset of the electrocardiogram training data.

In some aspects, the techniques described herein relate to a method, wherein the hypertension risk prediction includes a hypertension probability score that indicates a likelihood of the individual having hypertension and a hypertension risk stratification that categorizes the individual into risk groups based on the hypertension probability score compared to one or more thresholds.

In some aspects, the techniques described herein relate to a method, further including, generating auxiliary predictions based on the electrocardiogram feature map, the auxiliary predictions including at least one of an age prediction, a sex prediction, or a medication prediction.

In the following discussion, an example environment is first described that may employ the techniques described herein. Example implementation details and procedures are then described that may be performed in the example environment as well as other environments.

Consequently, performance of the example procedures is not limited to the example environment and the example environment is not limited to performance of the example procedures.

As used herein, the singular forms “a,” “an,” and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less from the specified value, insofar as such variations are appropriate to perform in the disclosed techniques. It is to be understood that the value to which the modifier “about” or “approximately” refers is itself also specifically disclosed.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets.

In the description of the figures, like numerals represent like (but not necessarily identical) elements throughout the figures.

### Example Environment

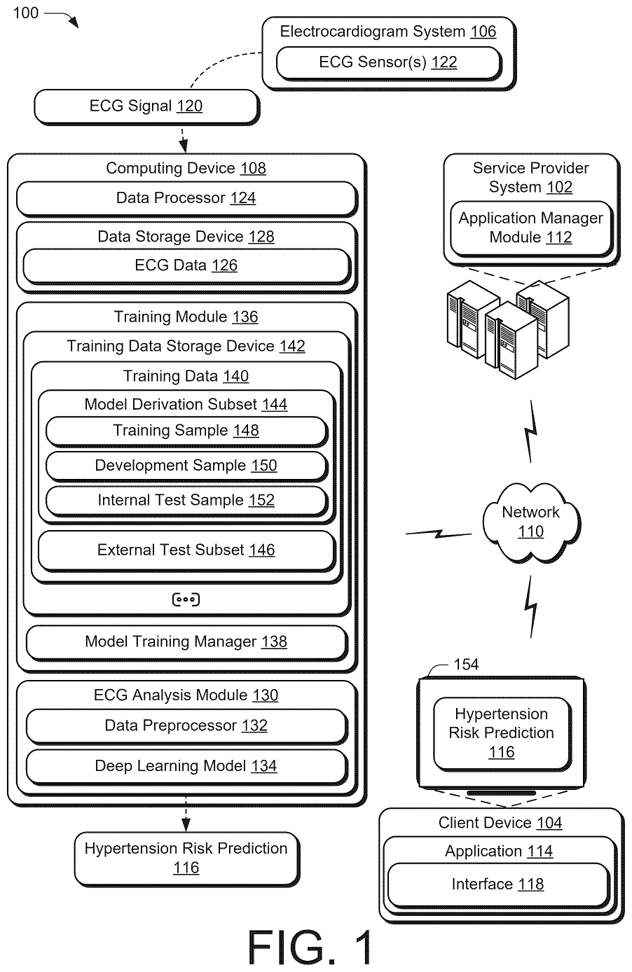

FIG. 1 is an illustration of an environment 100 in an example implementation that is operable to employ electrocardiogram-based deep learning for hypertension prediction as described herein. The illustrated environment 100 includes a service provider system 102, a client device 104, an electrocardiogram system 106, and a computing device 108 that are communicatively coupled, one to another, via a network 110. The network 110 may enable wired and/or wireless electronic communication, for example. Although the computing device 108 is illustrated as separate from the service provider system 102 and the client device 104, this functionality may be incorporated as part of the service provider system 102 and/or the client device 104, further divided among other entities, and so forth. By way of example, an entirety of or portions of the functionality of the computing device 108 may be incorporated as part of the service provider system 102 and/or the client device 104. Additionally or alternatively, an entirety of or portions of the client device 104 may be incorporated as part of the service provider system 102 and/or the computing device 108.

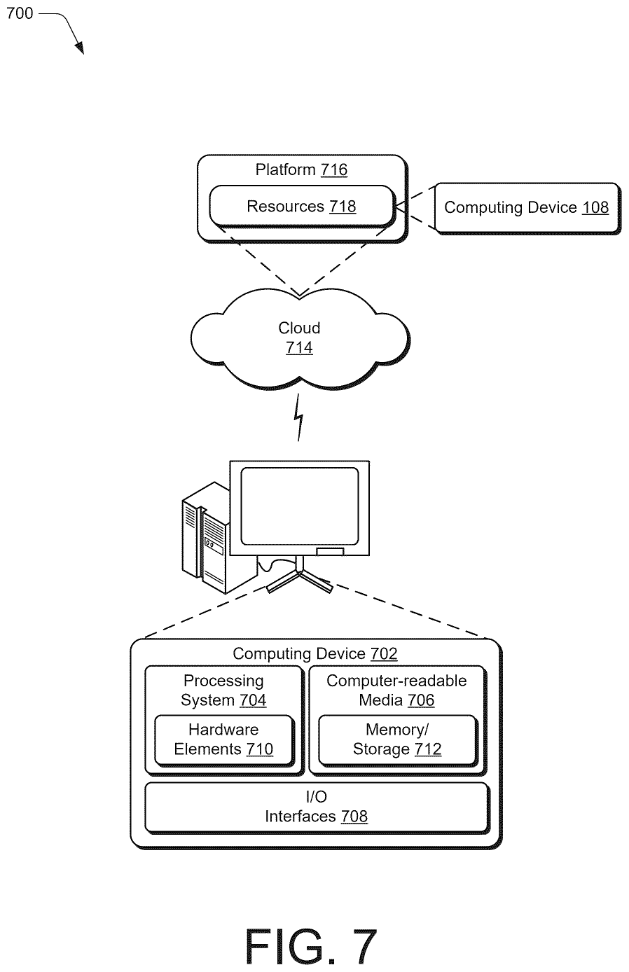

Computing devices that are usable to implement the service provider system 102, the client device 104, and the computing device 108 may be configured in a variety of ways. A computing device, for instance, may be configured as a desktop computer, a laptop computer, a mobile device (e.g., assuming a handheld configuration such as a tablet or mobile phone), and so forth. Thus, the computing device may range from full resource devices with substantial memory and processor resources (e.g., personal computers) to a low-resource device with limited memory and/or processing resources (e.g., mobile devices). Additionally, a computing device may be representative of a plurality of different devices, such as multiple servers utilized to perform operations “over the cloud,” as further described in relation to FIG. 7.

The service provider system 102 is illustrated as including an application manager module 112 that is representative of functionality to provide access to the computing device 108 to a user of the client device 104 via the network 110. The application manager module 112, for instance, may expose content or functionality of the computing device 108 that is accessible via the network 110 by an application 114 of the client device 104. The application 114 may be configured as a network-enabled application, a browser, a native application, and so on, that exchanges data with the service provider system 102 via the network 110. The data can be employed by the application 114 to enable the user of the client device 104 to communicate with the service provider system 102, such as to receive application updates and features when the service provider system 102 provides functionality to manage the application 114.

In the context of the described techniques, the application 114 includes functionality to train and/or use a machine learning model to analyze ECG data and output a hypertension risk prediction 116, as will be elaborated herein. By way of example, the hypertension risk prediction 116 may include a probability score (e.g., a hypertension probability score) indicating the likelihood of hypertension or risk of developing hypertension within a specified time frame. The probability score may be indicated as, for example, a numerical value (e.g., a value on a scale of 0 to 1) or a percentage (e.g., a percentage between 1 and 100%). Additionally, or alternatively, the hypertension risk prediction 116 may include an indication of hypertension severity, categorizing the risk as low, moderate, or high. The hypertension risk prediction 116 may further indicate specific ECG features or patterns that contributed to the risk assessment, providing clinicians with actionable insights. In some implementations, the hypertension risk prediction 116 may also include recommendations for follow-up tests or interventions based on the predicted risk level.

In the illustrated example, the application 114 includes an interface 118 that is implemented at least partially in hardware of the client device 104 for facilitating communication between the client device 104 and the computing device 108. By way of example, the interface 118 includes functionality to receive inputs to the computing device 108 from the client device 104 (e.g., from a user of the client device 104) and output information, data, and so forth from the computing device 108 to the client device 104, including the hypertension risk prediction 116.

The computing device 108 illustrated in FIG. 1 is further configured to receive an ECG signal 120 from the electrocardiogram system 106. The electrocardiogram system 106 includes ECG sensors 122 configured to detect electrical activity of the heart of a subject (e.g., a patient) during an ECG recording. By way of example, the ECG sensors 122 may include one or more electrodes that detect voltage differences on the skin surface resulting from the heart's electrical activity. ECG signals are typically in the range of millivolts; the size of each electrical wave is termed the amplitude, and the number of cardiac cycles per minute is the heart rate. For medical applications, the frequency content of ECG signals typically lies within the range of 0.05-150 Hz. This frequency content range characterizes the physiological components of the ECG signal and is distinct from the sampling frequency (e.g., rate) of the electrocardiogram system 106, which is generally higher, as further mentioned below. After the ECG sensors 122 detect the electrical signals from the body, these signals are amplified and filtered to produce the ECG signal 120. The ECG signal 120 may be in the form of a time-varying voltage signal, for instance.

The terms “record” or “recording” may be used herein to refer to acquiring data through the process of detecting and processing electrical signals from the heart. The term “data” may be used herein to refer to one or more datasets acquired with an electrocardiogram system, such as the electrocardiogram system 106. In at least one implementation, data acquired via the electrocardiogram system 106 is processed via a data processor 124 of the computing device 108 to generate ECG data 126, which may be stored in a data storage device 128. The ECG data 126 may comprise individual heartbeat waveforms as well as longer recordings, e.g., multi-lead ECG strips. The data storage device 128 may represent one or more databases and other types of storage capable of storing the ECG data 126. The data storage device 128 may also store a variety of other data, such as patient demographic information, electronic health record information, and so forth.

By way of example, the data processor 124 may process the ECG signal 120 in real-time during a recording session (e.g., a period of time where a healthcare provider acquires the ECG signal 120 via the electrocardiogram system 106), as the electrical signals are received and transmitted to the computing device 108. The term “real-time” is defined to include a procedure that is performed without intentional delay (e.g., substantially at the time of occurrence). In the context of electrocardiography, for instance, real-time denotes generating the ECG data 126 substantially as the ECG signal 120 is acquired. As a non-limiting example, the electrocardiogram system 106 may acquire data at a real-time sampling rate ranging between 250 and 1000 Hz. However, it should be understood that the real-time sampling rate may be dependent on the specific application and the amount of detail required. Accordingly, when acquiring a relatively large amount of data, the real-time processing may be adjusted. Thus, some implementations may have real-time sampling rates that are considerably faster than 1000 Hz, while other embodiments may have real-time sampling rates slower than 250 Hz. In at least one variation, the data may be stored temporarily in a buffer (not shown) during a recording session and processed in less than real-time by the data processor 124 in an off-line operation.

The ECG data 126 generated by the computing device 108 from the ECG signal 120 may be updated at a same or similar rate at which the ECG signal 120 is acquired. The data storage device 128 may store the processed ECG data 126. In at least one implementation, the ECG data 126 are stored in a manner to facilitate retrieval thereof according to its order or time of acquisition. The data storage device 128 may comprise any known data storage medium. It is to be appreciated that while the data processor 124 and the data storage device 128 are illustrated as part of the computing device 108, in at least one variation, the data processor 124 and/or the data storage device 128 are part of the electrocardiogram system 106 and/or another computing device.

In one or more implementations, the data processor 124 may process the ECG signal 120 in different analysis modules (e.g., QRS detection, rhythm analysis, ST segment analysis, QT interval measurement, and the like) to extract various features and measurements. When multiple ECG leads are obtained, the data processor 124 may also be configured to analyze the relationships between different leads. For example, one or more modules may perform signal filtering, baseline wander removal, QRS complex detection, heart rate calculation, arrhythmia detection, ST segment analysis, T wave alternans analysis, and the like, and combinations thereof. The modules may include, for example, a feature extraction module to identify points in the ECG waveform such as P waves, QRS complexes, and T waves. In ECG analysis, for instance, normal sinus rhythm may show a characteristic pattern of P waves, QRS complexes, and T waves, whereas various abnormalities may result in changes to this pattern. A display module may be provided that reads the ECG data 126 from the data storage device 128 and displays the ECG waveform or a derived measurement in real-time while a procedure (e.g., an ECG recording procedure) is being performed on the patient and/or after completion of the procedure.

Further, the components of the electrocardiogram system 106 and/or the computing device 108 may be coupled to one another to form a single structure, may be separate but located within a common room, or may be remotely located with respect to one another. For example, one or more of the modules described herein may operate in a data server that has a distinct and remote location with respect to other components of the electrocardiogram system 106 and/or the computing device 108, such as the ECG sensors 122. Optionally, the electrocardiogram system 106 may be a unitary system that is capable of being moved (e.g., portably) from room to room. For example, the electrocardiogram system 106 may include wheels, may be transported (e.g., on a cart), or may comprise a handheld device.

In at least one implementation, the ECG data 126, or a portion thereof, may be processed by an ECG analysis module 130. By way of example, the ECG analysis module 130 is representative of the functionality implemented at least partially in hardware of the computing device 108 to analyze the ECG data 126, such as one or more ECG recordings, and output the hypertension risk prediction 116. In the example shown in FIG. 1, the ECG analysis module 130 includes a data preprocessor 132 and a deep learning model 134 for analyzing the ECG data 126 to generate the hypertension risk prediction 116. The deep learning model 134 is a trained machine learning model. By way of example, the ECG analysis module 130 may include multiple different deep learning models that correspond to different types of machine learning models, where the underlying models learn using different approaches (e.g., supervised learning, unsupervised learning, and/or reinforcement learning), and/or multiple different deep learning models having a same model architecture but that are trained using different input data and/or to output a different type of hypertension risk prediction 116. By way of example, these models may include regression models (e.g., linear, polynomial, and/or logistic regression models), classifiers, neural networks, and reinforcement learning based models, to name just a few.

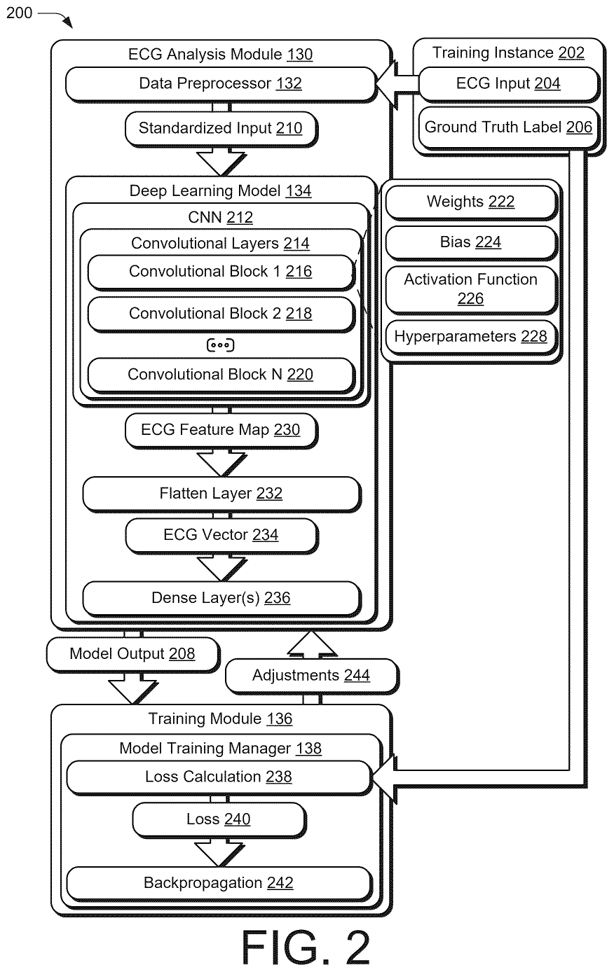

The deep learning model 134 may be configured as (or include) other types of models without departing from the spirit or scope of the described techniques. These different machine learning models may be built or trained (or the model otherwise learned), respectively, using different inputs and/or different algorithms due, at least in part, to different architectures and/or learning paradigms. Accordingly, it is to be appreciated that the following discussion of the functionality of the ECG analysis module 130 is applicable to a variety of machine learning models. For explanatory purposes, however, the functionality of the deep learning model 134 will be described generally with respect to a convolutional neural network (CNN). The CNN, for instance, may include one temporal dimension. By way of example, the deep learning model 134 may be based on a 1D CNN architecture to process temporal information in ECG signals. Additional details of the CNN will be described herein, e.g., with respect to FIG. 2. In one or more implementations, the CNN is combined with additional architectures and/or model portions to produce the hypertension risk prediction 116.

The computing device 108 further includes a training module 136 that is implemented at least partially in hardware of the computing device, at least in part, to deploy deep learning to generate the deep learning model 134. By way of example, the training module 136 includes a model training manager 138 that is configured to manage the deep learning model 134. This model management may include, for example, building the deep learning model 134, training the deep learning model 134, updating the model(s), and so forth. For instance, the model training manager 138 may be configured to carry out this model management using, at least in part, training data 140 maintained in a training data storage device 142. As illustrated in the environment 100 of FIG. 1, the training data 140 may include a model derivation subset 144 and an external test subset 146. For example, the model training manager 138 may use at least a portion of the model derivation subset 144 of the training data 140 as input for training the deep learning model 134 and may use at least a portion of the external test subset 146 for evaluating performance of the deep learning model 134 after the deep learning model 134 is at least initially trained. The model derivation subset 144 and the external test subset 146 may include ECG recordings from different data sources, for example. As such, the external test subset 146 may be used to verify that the deep learning model 134 achieves performance goals on data from diverse sources. Moreover, although the training data 140 may include multiple ECG recordings from a single patient, each separate recording may be treated as a separate training sample. Ellipses denote that more than one training data set may be stored in the training data storage device 142.

It is to be appreciated that although the model derivation subset 144 and the external test subset 146 are shown stored in the same training data storage device 142, in at least one variation, the model derivation subset 144 and the external test subset 146 are distributed among multiple storage locations. Alternatively, or in addition, the training data storage device 142 may be stored in a location that is external to the computing device 108 and accessed by the computing device 108 (e.g., over the network 110). As such, it is to be appreciated that the relative arrangement of the various modules and data storage devices in FIG. 1 is non-limiting, and variations are possible.

In one or more implementations, the model derivation subset 144 is further subdivided into a training sample 148, a development sample 150, and an internal test sample 152. By way of example, the training sample 148 may comprise a largest portion of the of the model derivation subset 144, while the development sample 150 and/or the internal test sample 152 may comprise a smallest portion of the model derivation subset 144. As a non-limiting example, the training sample 148 comprises 70% of the model derivation subset 144, the development sample 150 comprises 15% of the model derivation subset 144, and the internal test sample 152 comprises 15% of the model derivation subset 144, although other divisions are possible. The training sample 148, for instance, may comprise between 50% and 80% of the model derivation subset 144, the development sample 150 may comprise between 10% and 40% of the model derivation subset 144, and the internal test sample 152 may comprise between 5% and 30% of the model derivation subset 144.

Broadly speaking, the training sample 148 may be input to the deep learning model 134 during a training process, where the deep learning model 134 learns patterns and relationships in the data. During the training process, weights and parameters of the deep learning model 134 may be adjusted to reduce (e.g., minimize) errors between an output of the model and a ground truth label associated with a corresponding ECG recording (e.g., the hypertension risk prediction, as determined by a clinician). Following completion of the training process, the deep learning model 134 is able to accurately output the hypertension risk prediction 116 of the training sample 148.

The development sample 150 may be input to the deep learning model 134 during a model refinement (e.g., fine-tuning) process, where the deep learning model 134 is adjusted to prevent or reduce overfitting/underfitting of the model to the training sample 148. By way of example, the model refinement process may be performed following each round (or epoch) of training to evaluate how well the deep learning model 134 performs on data that is different from the training sample 148. During the model refinement process, for instance, a complexity, learning rate, and/or regularization of the deep learning model 134 may be adjusted (e.g., by the model training manager 138, automatically and/or based on user input) based on the performance of the deep learning model 134 with the development sample 150. As an illustrative example, if the deep learning model 134 accurately predicts the hypertension risk prediction 116 of the training sample 148 but not the development sample 150, overfitting of the deep learning model 134 to the training sample 148 is indicated. As such, the model refinement process enables settings of the deep learning model 134 and/or its training to be fine-tuned so that the deep learning model 134 can be generalized to unseen data (e.g., data that the deep learning model 134 has not been trained on).

The internal test sample 152 may be input to the deep learning model 134 during an internal validation process that is performed after the deep learning model 134 is trained and fine-tuned. The internal test sample 152 comprises data that was unseen by the deep learning model 134 during the training and model refinement processes described above but that is derived from the same dataset (e.g., the model derivation subset 144). The internal validation process evaluates the performance of the deep learning model 134 on similar data as to that used during the training and model refinement processes. If the deep learning model 134 does not meet acceptable or desired performance criteria (e.g., as defined by model developers) during the internal validation process, the deep learning model 134 may be returned to the training and/or model refinement processes so that changes can be made. For example, changes may be made to feature selection, the model architecture, regularization techniques, hyperparameter tuning, and the like.

The model training manager 138 may leverage the functionality of the data preprocessor 132 to process the training data 140 during the training, refinement, and validation processes described above. The data preprocessor 132, for instance, may remove patient identifying information and standardize the ECG data 126 to be input into the deep learning model 134, such as by normalizing, upsampling, and/or zero-padding the ECG data 126. As used herein, “normalizing” the ECG data 126 may refer to adjusting ECG voltage measurements to a standardized scale and/or range, which may include scaling voltages (e.g., to have a mean of zero and a standard deviation of one), adjusting amplitudes to a predetermined range, and/or other normalization techniques to ensure consistent formatting of the ECG data 126 prior to input into the deep learning model 134. The standardization performed by the data preprocessor 132 may ensure that the deep learning model 134 learns clinically relevant features associated with hypertension rather than variations in data acquisition parameters, recording equipment characteristics, and/or formatting differences between different electrocardiogram systems 106.

Once the deep learning model 134 is at least initially trained and internally validated, the model training manager 138 may use the external test subset 146 of the training data 140 to evaluate generalizability of the deep learning model 134. As mentioned above, the external test subset 146 comprises ECG data from a different data source, such as a different healthcare facility and/or patient population. By way of example, the external test subset 146 may be input into the deep learning model 134, and the deep learning model 134 may output the hypertension risk prediction 116 for respective ECG recordings. The hypertension risk prediction 116 may be compared to the ground truth labels to evaluate an accuracy of the deep learning model 134 on this novel dataset. The external test subset 146, for instance, may be used to verify that the performance of deep learning model 134 is not specific to the data source of the model derivation subset 144.

In response to the deep learning model 134 meeting desired or acceptable performance metrics, the deep learning model 134 may be deployed for determining the hypertension risk prediction 116 of newly obtained ECG data, including ECG data for which there is no ground truth label. By way of example, the ECG data 126 may correspond to ECG recordings that have not been evaluated by a clinician or technician with respect to the hypertension risk prediction 116. The ECG data 126 may be input into the (trained and validated) deep learning model 134 at or around the time of acquisition, and the deep learning model 134 may output the hypertension risk prediction 116 accordingly, thus enabling a streamlined ECG analysis workflow.

In at least one implementation, the hypertension risk prediction 116 includes a probability score indicating a likelihood of hypertension or risk of developing hypertension within a specified time frame. Additionally, or alternatively, the hypertension risk prediction 116 may include an estimated time frame for potential hypertension onset, which could range from months to years. In at least one implementation, the hypertension risk prediction 116 additionally or alternatively includes an indication of hypertension severity, which may categorize the risk as low, moderate, or high. The hypertension risk prediction 116 may further indicate specific ECG features or patterns that contributed to the risk assessment, providing clinicians with actionable insights. In some implementations, the hypertension risk prediction 116 may also include recommendations for follow-up tests or interventions based on the predicted risk level.

Although the above discussion is focused on inputting the ECG data 126 to the deep learning model 134 and receiving the hypertension risk prediction 116 as the output, it is to be appreciated that additional inputs and/or outputs are possible. By way of example, the deep learning model 134 may be trained on auxiliary (e.g., secondary) tasks that may help the deep learning model 134 learn shared representations of the input data. The learning of shared representations may increase the performance of the deep learning model 134 for the hypertension risk prediction 116. Examples of auxiliary tasks include predicting age, sex, and the use of antihypertensive medications.

The client device 104 is shown displaying, via a display device 154, the hypertension risk prediction 116. Alternatively, or in addition, the client device 104 may display, via the display device 154, the ECG data 126. It is to be appreciated that the hypertension risk prediction 116 may be also stored in a memory of the computing device 108 and/or the client device 104 for subsequent access.

In this way, the ECG analysis module 130 enables automated ECG analysis for identifying and/or monitoring hypertension risk, which may be used in patient stratification, risk assessment, and/or treatment monitoring.

### Electrocardiogram-Based Deep Learning for Hypertension Prediction

FIG. 2 depicts an example training process 200 that may be used in generating the deep learning model 134 of the ECG analysis module 130 of FIG. 1. It is to be appreciated that the example training process 200 denotes one implementation of a training process that may be used in generating at least a portion of the deep learning model 134.

In the example training process 200, a training instance 202 includes an ECG input 204 and a ground truth label 206 associated with the ECG input 204. The ECG input 204, for instance, includes a sequence of voltage measurements obtained during a single acquisition process. In at least one implementation, the ECG input 204 comprises a 12-lead ECG. In at least one variation, the ECG input 204 may comprise a single-lead ECG, a 3-lead ECG, a 5-lead ECG, a 6-lead ECG, or another lead configuration. The specific lead configuration used may be selected based on factors such as the desired analysis, available equipment, or patient characteristics. During the training, the ECG input 204 is part of a corresponding portion of the training data 140 (e.g., of the training sample 148).

Each ECG input 204 may be separately evaluated by the deep learning model 134 to generate a model output 208 (e.g., an output produced by the deep learning model 134), which corresponds to the single ECG input 204. As such, the training instance 202 includes an input portion (e.g., the ECG input 204) and an associated expected output portion (e.g., the ground truth label 206), and a great many training instances 202 may be used during the training process. The model output 208 corresponds to a prediction for a particular classification and/or measurement task(s) the deep learning model 134 is being trained for. By way of example, the deep learning model 134 may be trained to output (e.g., as the model output 208) the hypertension risk prediction 116. Additionally, the model may be trained on auxiliary tasks such as predicting age, sex, and/or the use of antihypertensive medications. The ground truth label 206 defines a true or expected output of the deep learning model 134 for the ECG input 204. By way of example, the ground truth label 206 may be or may be derived from clinical labels and/or adjudicated by an expert for comparison to the model output 208 during training.

In at least one implementation, the ground truth label 206 includes a hypertension classification. The hypertension classification may be a binary value, where the presence of hypertension is denoted by a one, and the absence of hypertension is denoted by a zero. The presence of hypertension may be based on one or more or a combination of a baseline diagnosis of hypertension by International Classification of Diseases (ICD) 9 or ICD-10 codes, a baseline systolic blood pressure of at least 140 mmHg, and a baseline diastolic blood pressure of at least 90 mmHg. Multiple ECGs may be used per patient during training, including ECGs before and after the start of follow up. It is to be appreciated that the ground truth label 206 may further include additional labels, such as those corresponding to auxiliary training tasks (e.g., sex, age, and the use of antihypertensive medications).

In the example shown in FIG. 2, the ECG input 204 is provided to the data preprocessor 132, which generates a standardized input 210 (e.g., a standardized version of the ECG input 204). As mentioned above with respect to FIG. 1, the data preprocessor 132 may remove patient identifying information and standardize ECG data that is to be input into the deep learning model 134. For example, the data preprocessor 132 may apply filtering techniques to remove noise and baseline wander from the ECG signal. The data preprocessor 132 may also normalize the amplitude of the ECG waveforms to a standard range. Additionally, or alternatively, the data preprocessor 132 may truncate or extend the ECG data to a predetermined time duration (e.g., 10 seconds). By way of example, ECG data with shorter durations than the predetermined time duration may be zero-padded to contain a standardized number of measurements. The data preprocessor 132 may upsample or downsample data to a predetermined frequency (e.g., 500 Hz). By way of example, data recorded at 250 Hz may be upsampled to 500 Hz. The data preprocessor 132 may also handle missing or corrupted data points through interpolation or other imputation methods, such as zero-padding. The preprocessing performed by the data preprocessor 132 may thus be used to provide consistent, high-quality inputs to the deep learning model 134, which may improve the performance and generalizability of the deep learning model 134 across different ECG recording conditions and equipment. As a non-limiting example, the standardized input 210 comprises a time-series of 5000 voltage measurements for each of the 12 leads, sampled at 500 Hz over 10 seconds.

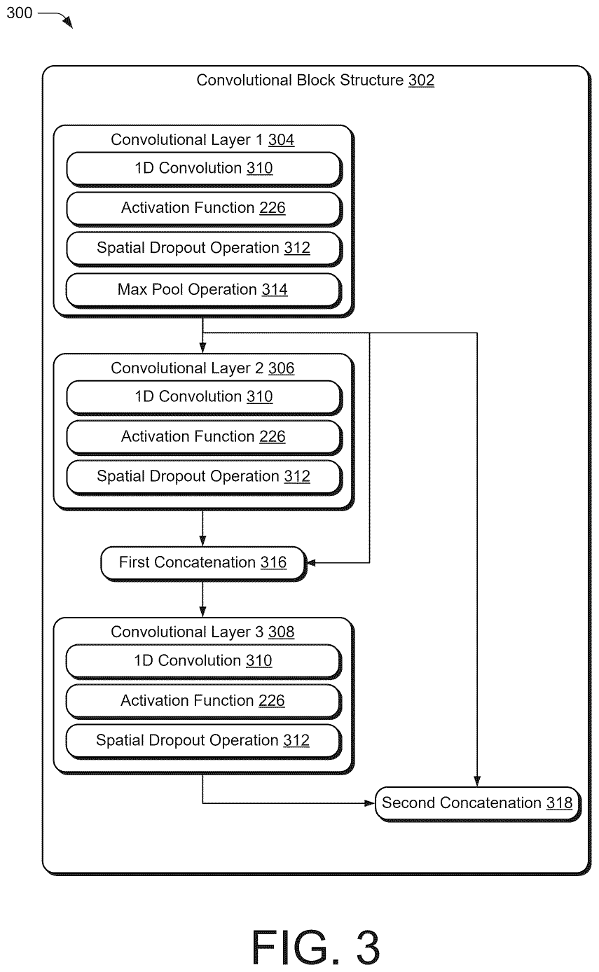

The data preprocessor 132 may be operatively connected to the deep learning model 134 to provide the standardized input 210 to the deep learning model 134. In the example training process 200 shown in FIG. 2, the deep learning model 134 comprises a CNN 212 having convolutional layers 214. The convolutional layers 214 are shown as including a first convolutional block 216 (e.g., “convolutional block 1”), a second convolutional block 218 (e.g., “convolutional block 2”), and an N^{th} convolutional block 220 (e.g., “convolutional block N”), where N is an integer representing the total number of convolutional blocks. Ellipses denote that additional convolutional blocks may be present between the second convolutional block 218 and the N^{th} convolutional block 220. As a non-limiting example, there may be four convolutional blocks. In general, the convolutional layers 214 may be configured to extract features of the standardized input 210. An example architecture of the convolutional blocks is described below with respect to FIG. 3.

As used herein in the context of machine learning, the term “features” may refer to individual measurable properties, characteristics, and/or patterns of the input data that are used by the deep learning model 134 to make predictions or decisions. Features may be numerical values extracted or computed from raw data and/or a standardized version of the raw data, such as an electrocardiogram signal, that represent relevant aspects of the data for the task at hand. Initial features, for instance, may be values from the standardized input 210 itself, often represented as voltage readings over time. Each time step in the standardized input 210 is a potential feature that reflects the heart's electrical activity at that instant. Extracted features (e.g., extracted by the CNN 212) may include, for example, basic shapes or waveforms in the ECG, like peaks or troughs; temporal patterns in the ECG that may reflect heartbeat rhythms or irregularities; and/or combinations of patterns that might correspond to higher-level physiological or health indicators. In the deep learning model 134, features serve as the basis for learning data patterns and relationships, allowing the deep learning model 134 to generalize from training data to make predictions on new, unseen data.

In the present example shown in FIG. 2, the convolutional layers 214 are grouped together in blocks. By way of example, a block may comprise multiple convolutional layers that are stacked together with operations such as pooling, batch normalization, and/or activation functions to form a unit that is able to extract more complex or abstract features than a single convolutional layer alone. In at least one variation, one or more of the convolutional layers 214 are arranged as single convolutional layers. For instance, the first convolutional block 216, the second convolutional block 218, and/or the Nth convolutional block 220 instead may be a single convolutional layer. In at least one implementation, the CNN 212 is a dense convolutional network, where each block connects to all subsequent blocks, allowing features from earlier blocks to be directly accessible to later layers.

The first convolutional block 216, for example, may apply a kernel (e.g., a filter) to the standardized input 210, which scans across the dimension to extract low-level features like peaks, valleys, and edges across the ECG signal. The features detected by the first convolutional block 216 may be fed to the subsequent convolutional layers 214 (e.g., the blocks) in sequence, allowing the CNN 212 to detect increasingly complex patterns in the standardized input 210. Accordingly, each block may progressively learn more complex features, helping the model understand the ECG data at multiple scales. By way of example, the second convolutional block 218 may learn higher-level representations, like specific morphologies of waveforms, variations in wave amplitude, or frequency patterns, and the Nth convolutional block 220 may learn global patterns that span across multiple heartbeats. Alternatively, or in addition, the CNN 212 may perform progressive downsampling, where the number of input measurements decreases through striding or pooling operations. This may include reducing the temporal dimension to enable the deep learning model 134 to focus on capturing larger patterns and relationships in the data over longer time spans, while reducing the computational load.

The first convolutional block 216, as well as others of the convolutional layers 214, may comprise weights 222, a bias 224, an activation function 226, and hyperparameters 228. By way of example, the weights 222 and the bias 224 may be randomly initialized and then “learned” during the training process, as elaborated below. The CNN 212, for instance, performs a series of convolutions. A convolution is a mathematical operation where the kernel slides over an input ECG signal and performs element-wise multiplication with the values of the signal at each temporal position. The results are summed up to produce a single output value for that temporal position, and this process is repeated across the ECG signal (e.g., the standardized input 210) to produce a feature map. The kernel comprises a matrix of numbers, which are the weights 222 of the kernel, that is applied to the input. The bias 224 is a single number added to the result of the convolution. After each convolution, the feature map may be passed through the activation function 226, which may introduce non-linearity. In at least one implementation, the activation function 226 is a swish activation function. After several convolution operations, a pooling layer may be used to reduce the size of the feature map.

The hyperparameters 228 are not learned during the training process but can be adjusted to increase performance. The hyperparameters 228 may comprise depth, stride, and zero-padding. Depth controls the number of neurons within a given convolutional layer of the convolutional layers 214. Reducing the depth may increase the speed of the CNN 212 but may also reduce the accuracy of the CNN 212. Stride determines how much the kernel moves or “slides” across the input. A stride of two means that the kernel moves two positions along the ECG signal at each step, effectively skipping one sample each time. A higher stride value would thus enable the CNN 212 to process data more quickly and reduces the spatial dimensions of the feature map. Zero-padding adds zeros around the edges of the input signal, allowing the kernel to be applied to edge samples where it would otherwise not fit, and ensuring that the output of the convolution remains the same size as the input when desired.

In at least one implementation, the CNN 212 outputs an ECG feature map 230, which may be a multi-dimensional representation of the features extracted from the standardized input 210. The ECG feature map 230 may capture various characteristics of the ECG signal at different levels of abstraction, from low-level features like signal peaks and valleys to higher-level patterns such as waveform morphologies and rhythm irregularities. The ECG feature map 230 may be processed through a flatten layer 232, which may transform the ECG feature map 230 into an ECG vector 234. The ECG vector 234 may be a one-dimensional vector that summarizes the features or characteristics of the standardized input 210 that have been learned by the CNN 212. In some implementations, the dimensionality of the ECG vector 234 may be reduced compared to the ECG feature map 230, which may help in managing computational complexity while retaining information for hypertension prediction.

The ECG vector 234 is input into one or more dense layers 236, which are fully connected layers where every neuron (node) is fully connected to every neuron in the previous and next layer. The one or more dense layers 236 may function to aggregate information learned by previous layers and make final predictions for the model output 208. Accordingly, the one or more dense layers 236 may comprise an output layer (or final layer) of the deep learning model 134. The one or more dense layers 236 may learn complex relationships between the features represented in the ECG vector 234, which may enable the model to capture subtle patterns that may be indicative of hypertension risk, for instance. Similar to the convolutional layers 214, the one or more dense layers 236 may comprise the weights 222, the bias 224, the activation function 226, and the hyperparameters 228. It is to be appreciated that values of at least a portion of these parameters and/or a type of activation function used are specific to a given convolutional layer 214 or dense layer 236. The one or more dense layers 236, for instance, may take the ECG vector 234 as an input vector, multiply it by a matrix of the weights 222, add the bias 224, and then apply the activation function 226 to produce the model output 208.

The model output 208 is received by the model training manager 138, which may perform a loss calculation 238. The loss calculation 238 may use a loss function to compute the difference between the model output 208 and the ground truth label 206, e.g., a loss 240. The loss 240, for instance, is a measure of the error of the deep learning model 134 in determining the model output 208. Various types of loss functions may be used depending on the specific task and model architecture. For classification tasks, cross-entropy loss may be employed. For regression tasks, mean squared error or mean absolute error may be used.

A goal of the training is to minimize the loss 240 by adjusting the weights 222 and biases 224 of the given deep learning model of the deep learning model 134. In order to do so, the model training manager 138 may employ backpropagation 242 to compute how the parameters are to be updated based on a gradient of the loss 240 with respect to each parameter. By way of example, the backpropagation 242 may utilize an optimization algorithm such as gradient descent (e.g., Adam). The backpropagation 242 results in adjustments 244, which are used to update the weights 222 and biases 224 of the deep learning model 134.

As such, following many rounds of training with a large number of training instances 202, the model output 208 becomes consistent with the ground truth label 206 due to the deep learning model 134 “learning” to minimize the loss 240 between the model output 208 and the ground truth label 206.

In this way, the training process 200 provides a comprehensive framework for developing and refining the deep learning model 134 for the hypertension risk prediction 116. This approach may enable efficient learning from large datasets of ECG recordings, which may improve the accuracy and generalizability of the hypertension risk prediction 116. The trained deep learning model 134 is a specialized machine learning model that may be deployed in various clinical settings to analyze newly acquired ECG data from patients. For example, in a hospital or primary care clinic, the deep learning model 134 could be integrated into existing ECG analysis workflows. When a patient undergoes an ECG recording, the resulting ECG data may be automatically processed by the ECG analysis module 130, providing an immediate hypertension risk assessment. This real-time analysis may assist healthcare providers in several ways. As one example, patients at high risk of hypertension who may benefit from additional diagnostic tests or preventive interventions may be identified. For patients with known hypertension, the hypertension risk prediction 116 may aid in monitoring disease progression and assessing the effectiveness of current treatments. As another example, the ability of the deep learning model 134 to perform auxiliary tasks, such as predicting age, sex, and use of antihypertensive medications, may offer additional clinical value. These predictions could serve as a quality control measure, potentially flagging discrepancies in patient information or ECG lead placement errors. Such checks may enhance the overall reliability of the ECG analysis process. Moreover, in emergency settings, the rapid risk assessment provided by the hypertension risk prediction 116 could support triage decisions, helping to prioritize patients who may require urgent cardiovascular care. This could be particularly valuable in resource-constrained environments or during high-volume periods.

FIG. 3 illustrates an example implementation 300 of a convolutional block structure 302 that may be used in at least a portion of the convolutional layers 214 of the CNN 212 of FIG. 2. The convolutional block structure 302 may include multiple convolutional layers and operations configured to extract features from the input ECG data.

In the implementation 300, the convolutional block structure 302 includes a first convolutional layer 304 (e.g., “convolutional layer 1”), a second convolutional layer 306 (e.g., “convolutional layer 2”), and a third convolutional layer 308 (e.g., “convolutional layer 3”). The first convolutional layer 304 may include a 1D convolution 310, which applies a one-dimensional convolution operation to the input data. The output of the 1D convolution 310 may be passed through an activation function 226, which allows the CNN 212 to model complex relationships in the data that a purely linear transformation would miss. Following the activation function 226, a spatial dropout operation 312 may be applied to reduce overfitting by randomly setting a fraction of input units to zero during training. The spatial dropout operation 312 may improve generalization of the CNN 212, for example. The first convolutional layer 304 may conclude with a max pool operation 314, which reduces the spatial dimensions of the feature maps and helps to achieve translation invariance. By way of example, the max pool operation 314 may divide the input into pooling regions and output the maximum value for each such region. This process may retain the most prominent features, thereby making the output of the first convolutional layer 304 less sensitive to small translations or shifts in the input ECG signal. This property may enable the CNN 212 to maintain consistent performance even when ECG features, such as QRS complexes or T waves, appear at different time points across various ECG samples.

The output from the first convolutional layer 304 may be input into the second convolutional layer 306. The second convolutional layer 306 may also include the 1D convolution 310 followed by the activation function 226 and the spatial dropout operation 312. The second convolutional layer 306 may not include the max pool operation 314, at least in one implementation. This may allow the second convolutional layer 306 to maintain the spatial dimensions of its input, potentially preserving more fine-grained features of the ECG signal. In some examples, omitting the max pool operation 314 in the second convolutional layer 306 may enable the CNN 212 to capture more complex, higher-level features. Additionally, or alternatively, this approach may help in retaining the full resolution of the features extracted by the first convolutional layer 304, which may be beneficial for detecting subtle ECG abnormalities associated with hypertension risk.

The outputs from the first convolutional layer 304 and the second convolutional layer 306 may be combined in a first concatenation 316. The first concatenation 316 may allow the network to preserve and utilize features from both the first convolutional layer 304 and the second convolutional layer 306, which may capture both low-level and higher-level features of the ECG signal.

The concatenated output may be processed by the third convolutional layer 308, which follows a similar structure to the second convolutional layer 306, with the 1D convolution 310, the activation function 226, and the spatial dropout operation 312. Similar to the second convolutional layer 306, the third convolutional layer 308 may not include the max pool operation 314.

The output from the third convolutional layer 308 may be combined with the output from the first convolutional layer 304 in a second concatenation 318. The second concatenation 318 may allow the convolutional block structure 302 to maintain a direct connection to the earliest features extracted from the input, which may help in preserving low-level information throughout the convolutional block structure 302.

The convolutional block structure 302 incorporates skip connections in the first concatenation 316 and the second concatenation 318. These skip connections may enable information from earlier levels of the convolutional block structure 302 to be preserved in the final output. This may provide a multi-scale representation of the input data and may help gradients flow more effectively during the backpropagation 242. Moreover, concatenating features from different layers in the convolutional block structure 302 may enhance feature richness.

The specific configuration of the convolutional block structure 302 may be adjusted based on the characteristics of the ECG data and the hypertension prediction task. For example, the number of filters in each convolutional layer, the size of the convolution kernels, the dropout rate, and the pooling size may be tuned to optimize performance. Additionally, multiple instances of this convolutional block structure 302 may be stacked to form deeper networks, which may allow for the extraction of more complex and abstract features from the ECG data.

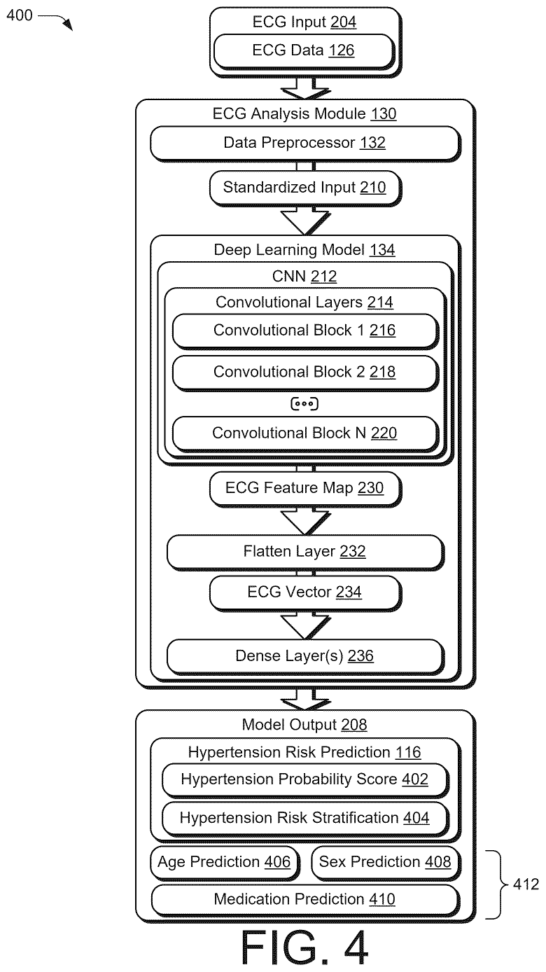

FIG. 4 depicts an example implementation 400 of using the deep learning model 134 to assess electrocardiogram data for hypertension prediction. Components previously introduced in FIGS. 1-3 are numbered the same and function as previously described.

The implementation 400 includes processing newly acquired ECG data 126 from an individual for which there is no ground truth label 206 via the ECG analysis module 130 to generate the hypertension risk prediction 116. As such, the ECG data 126, or a portion thereof, comprise the ECG input 204 in the implementation 400. The ECG data 126 are fed into the ECG analysis module 130, where the data preprocessor 132 generates the standardized input 210. This standardized input 210 is processed by the deep learning model 134, which includes the CNN 212 with the convolutional layers 214. As described with respect to FIGS. 2 and 3, for instance, these convolutional layers may include multiple blocks that progressively extract features from the ECG signal, such as waveform morphologies, rhythm patterns, and temporal relationships.

The CNN 212 outputs the ECG feature map 230, representing a high-dimensional abstraction of the input ECG data 126. The ECG feature map 230 is transformed by the flatten layer 232 into the one-dimensional ECG vector 234. The ECG vector 234 is processed through the one or more dense layers 236, which may learn complex non-linear relationships between the extracted features to produce the model output 208.

In the implementation 400, the model output 208 includes several predictions derived from the learned ECG features. The primary output is the hypertension risk prediction 116, which in this example includes a hypertension probability score 402 and a hypertension risk stratification 404. In at least one variation, the hypertension risk prediction 116 includes the hypertension probability score 402 or the hypertension risk stratification 404. The hypertension probability score 402 may include a probability or risk score for the individual having or developing hypertension. The individual may be stratified into risk categories using the hypertension risk stratification 404 (e.g., into high risk versus low risk, tertiles of risk, quintiles of risk, etc.) based on the hypertension probability score 402 compared to one or more thresholds. As a non-limiting example, the threshold is 0.85. For example, the individual may be considered low risk (e.g., as output via the hypertension risk stratification 404) in response to the hypertension probability score 402 being less than 0.85, whereas the individual may be considered high risk in response to the hypertension probability score 402 being greater than or equal to 0.85. In variations, however, the threshold is another value, such as a value in a range between 0.4 and 0.95. In at least one implementation, the threshold (or thresholds, when more than two risk categories are used) is set during the training process using the ground truth labels 206 regarding low risk versus high risk individuals.

In at least one implementation, the model output 208 includes additional predictions. By way of example, an age prediction 406 may estimate the biological age of the individual based on the ECG data 126, a sex prediction 408 may estimate the biological sex of the individual based on the ECG data 126, and/or a medication prediction 410 may estimate the likelihood of antihypertensive medication use based on the ECG data 126. These additional predictions (e.g., the age prediction 406, the sex prediction 408, and the medication prediction 410) are examples of auxiliary predictions 412. An “auxiliary prediction” may refer to a secondary or supplementary output generated by a machine learning model (e.g., the deep learning model 134) in addition to a primary prediction task or goal (e.g., the hypertension risk prediction 116). An auxiliary prediction may be produced by the same model architecture that generates the primary prediction, utilizing shared learned representations from the input data. With respect to the implementation 400, the auxiliary predictions 412 may be used to enhance the performance of the hypertension risk prediction 116 through multi-task learning, provide additional clinical context, and/or serve as quality control measures for validating the model output 208 against known patient information. The auxiliary predictions 412, for instance, may be produced by additional output nodes in the one or more dense layers 236 or by separate branches of the CNN 212 that share early layers with the hypertension risk prediction 116 task. In at least one implementation, the deep learning model 134 is trained to simultaneously produce these multiple outputs, which may enable the deep learning model 134 to leverage shared representations learned from the ECG data (e.g., the training data 140). This multi-tasking learning approach may improve the generalization and performance on the hypertension risk prediction 116 task by leveraging correlations between related cardiovascular outcomes and patient characteristics, for instance.

Additional example details of the usage of the model output 208 are described herein with respect to the Example Application.

Having discussed example details of the techniques for electrocardiogram-based deep learning for hypertension prediction, consider now example procedures to illustrate additional aspects of the techniques.

### Example Procedures

This section describes example procedures for electrocardiogram-based deep learning for hypertension prediction in one or more implementations. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations, at least a portion of the procedure is performed by a suitably configured device, such as the computing device 108 of FIG. 1, by executing instructions stored in a non-transitory computer-readable storage medium.

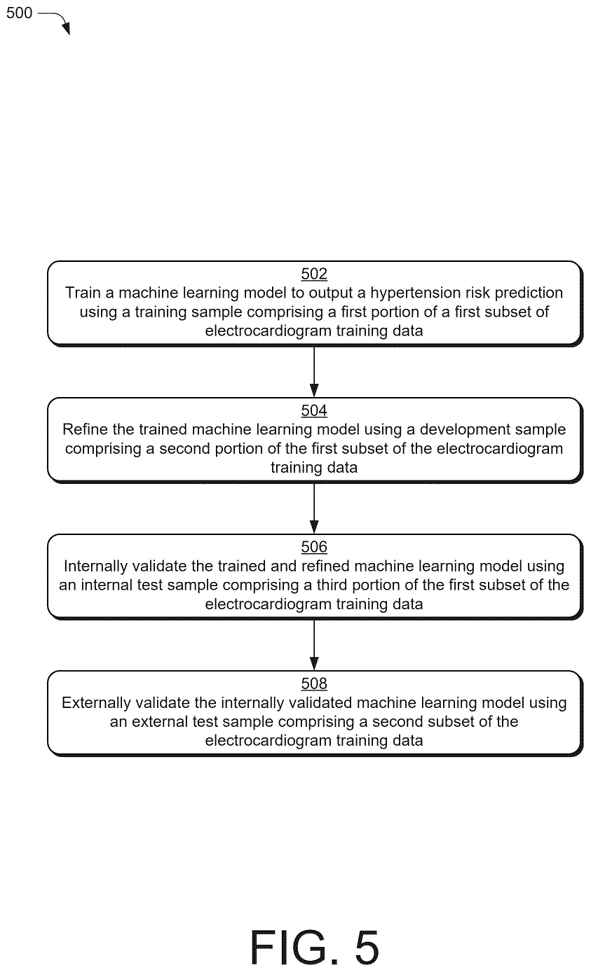

FIG. 5 depicts an example procedure 500 for training and validating a machine learning model to output a hypertension risk prediction according to one or more implementations. Where appropriate, reference will be made to components previously introduced in FIGS. 1-4.

A machine learning model is trained to output a hypertension risk prediction using a training sample comprising a first portion of a first subset of electrocardiogram training data (block 502). By way of example, the machine learning model may be included in the deep learning model 134 introduced with respect to FIG. 1. In one or more implementations, the machine learning model may include the CNN 212 with multiple convolutional layers 214 for extracting features from standardized ECG inputs. The training module 136 may train the machine learning model using the training sample 148 of the model derivation subset 144 of the training data 140.

In at least one implementation, the training process includes exposing the machine learning model to a diverse set of ECG data from the model derivation subset 144, allowing the machine learning model to “learn” patterns in the ECG data that are associated with a specific training task. For instance, the machine learning model “learns” via the adjustment of the weights 222 and biases 224, which may be adjusted through backpropagation 242 based on the loss 240 between the model output 208, e.g., corresponding to the hypertension risk prediction 116 output by the machine learning model for an ECG input (e.g., the ECG input 204 of the training instance 202), and the ground truth labels 206. This iterative process may continue until the model achieves satisfactory performance on the model derivation subset 144 of the training data 140.

In at least one implementation, as depicted in FIG. 4, the machine learning model may be trained to output the hypertension risk prediction 116, which may include the hypertension probability score 402. The model may be trained to output the hypertension probability score 402 as a likelihood and/or probability of the individual having and/or developing hypertension. As such, the ground truth labels 206 may include known hypertension outcomes for the ECG data 126 in the training data 140. Given this, the model training manager 138 may form a training instance that includes an input portion corresponding to a given ECG and an associated output portion with a hypertension outcome.

In at least one implementation, the model may be trained to produce the auxiliary predictions 412 in addition to the hypertension risk prediction 116. The auxiliary predictions 412 may include the age prediction 406, which may estimate the biological age of the individual; the sex prediction 408, which may estimate the biological sex of the individual; and the medication prediction 410, which may estimate the likelihood of antihypertensive medication use.

The trained machine learning model is refined using a development sample comprising a second portion of the first subset of the electrocardiogram training data (block 504). By way of example, the model training manager 138 may use the development sample 150 to fine-tune the model hyperparameters 228 and adjust the model weights 222 and biases 224. During the refinement process, the machine learning model is exposed to new ECG data from the model derivation subset 144, which is not seen by the machine learning model during the initial training process (e.g., as performed at block 502). This process helps identify and correct any overfitting that may have occurred during the training process. The hyperparameters 228, such as the learning rate, batch size, and/or regularization strength, may be adjusted to achieve a desirable performance on the development sample 150 while maintaining good generalization to unseen data, for instance.

The trained and refined machine learning model is internally validated using an internal test sample comprising a third portion of the first subset of the electrocardiogram training data (block 506). By way of example, the model performance may be evaluated on the internal test sample 152 to assess generalization. The internal validation process may provide an initial measure of an ability of the machine learning model to make accurate hypertension risk predictions on data from a same data source as the training sample 148 and the development sample 150 (e.g., the model derivation subset 144) but that was not used during the training (block 502) or refinement (block 504) processes described above.

During the internal validation process, for instance, outputs of the machine learning model may be compared against the ground truth label 206 for the internal test sample 152. Various performance metrics, such as accuracy, precision, recall, and F1 score for classification tasks, or mean absolute error and root mean squared error for regression tasks, may be calculated. The internal validation process may help identify any potential issues with the performance of the machine learning model and guide further refinement if the performance is not adequate.

The internally validated machine learning model is externally validated using an external test sample comprising a second subset of the electrocardiogram training data (block 508). By way of example, in response to the machine learning model meeting an acceptable or desired performance criteria, an external validation process may be performed using the external test subset 146. During the external validation process, the external test subset 146 may be input to the internally validated machine learning model. The external test subset 146 may be used to verify that the performance of deep learning model 134 is not specific to the data source of the model derivation subset 144. The external test subset 146, for instance, may comprise ECG data from one or more separate cohorts, often collected from a different institution and/or different patient population than the model derivation subset 144. The external validation process may provide an evaluation of the machine learning model with respect to a real-world scenario and may help identify biases or limitations that were not apparent in the internal validation. The external validation may provide confidence in the ability of the machine learning model to make accurate hypertension risk predictions across diverse patient populations and clinical settings.

In this way, the procedure 500 enables generation of a machine learning model that is able to output an accurate hypertension risk prediction 116 from an input ECG. By leveraging the full ECG waveform data, the machine learning model “learns” how to interpret latent information from ECGs that may not be readily interpretable by human observers. The machine learning model may learn to identify complex patterns that go beyond what is physically/manually measurable. These features, captured in the ECG feature map 230, represent a high-dimensional abstraction of the ECG data. By leveraging this information, the machine learning model may learn to make accurate predictions about hypertension risk that may not be evident from visual inspection or manual measurement performed by a human.

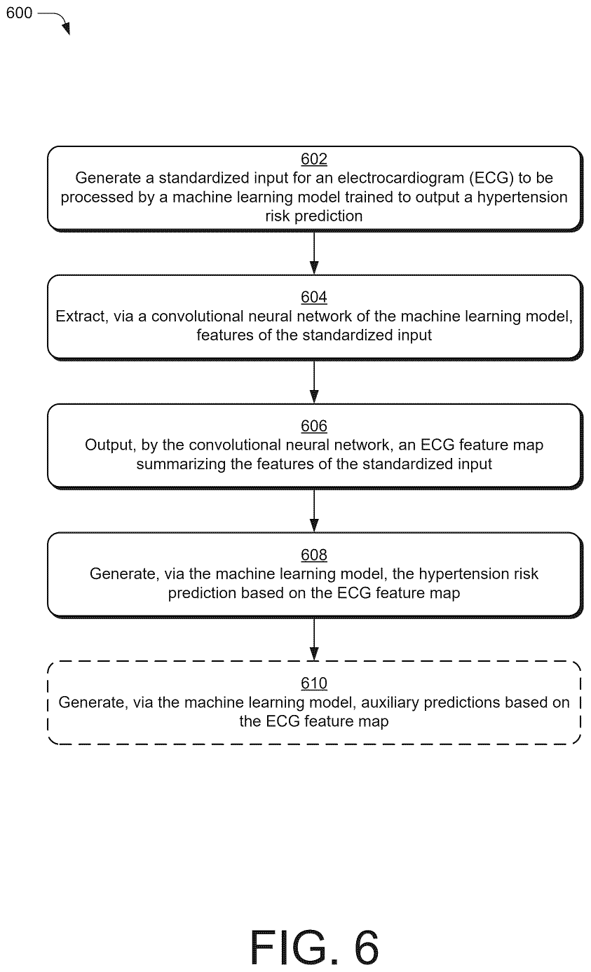

FIG. 6 depicts an example procedure 600 for generating a hypertension risk prediction from electrocardiogram data using a machine learning model according to one or more implementations. Where appropriate, reference will be made to components previously introduced in FIGS. 1-5.

A standardized input is generated for an electrocardiogram (ECG) to be processed by a machine learning model trained to output a hypertension risk prediction (block 602). By way of example, the data preprocessor 132 may generate the standardized input 210 from the ECG input 204, which corresponds to the ECG data 126 recorded from an individual. The ECG input 204, for instance, may be a single 12-lead ECG. The standardized input 210 may be processed to have consistent dimensions, sampling rate, and other characteristics with respect to other instances of the ECG input 204 to ensure uniform input to the machine learning model. Moreover, data overlays and non-ECG data (e.g., patient information, text) may be removed to generate the standardized input 210.

Features of the standardized input are extracted via a convolutional neural network of the machine learning model (block 604). By way of example, the machine learning model (e.g., the deep learning model 134) may utilize the CNN 212 to extract features from the standardized input 210. These features may represent various aspects of cardiac electrical activity depicted in the standardized input 210. The particular features extracted depend on the specific task(s) for which the machine learning model is trained. By way of example, during training, if a particular feature helps to minimize the loss 240, the training may reinforce this feature by adjusting the corresponding weights 222 and bias 224. In contrast, a feature that does not contribute to reducing the loss 240 may not be emphasized.

As such, the CNN 212 may be trained to extract task-relevant features such that the extracted features may vary from model to model of the deep learning model 134. For instance, the machine learning model trained to output the hypertension risk prediction 116, such as in the implementation 400 of FIG. 4, may extract features relevant to predicting hypertension risk.

The convolutional neural network outputs an ECG feature map summarizing the features of the standardized input (block 606). By way of example, the CNN 212 may output the ECG feature map 230, which summarizes the extracted features in a compact representation. The ECG feature map 230, for instance, may capture both spatial and temporal information to distill the information of the standardized input 210 into a lower-dimensional, meaningful representation that can be used for downstream tasks like classification or regression.

The machine learning model generates the hypertension risk prediction based on the ECG feature map (block 608). By way of example, the ECG feature map 230 may be processed through the flatten layer 232 to produce the ECG vector 234. The ECG vector 234 may then be fed to one or more dense layers 236, which output the hypertension risk prediction 116. In at least one implementation, such as depicted in FIG. 4, the one or more dense layers 236 may generate, as the hypertension risk prediction 116, the hypertension probability score 402 based on the ECG vector 234. The hypertension probability score 402 may indicate the likelihood the individual has hypertension or is at risk of developing hypertension. Additionally, the model output 208 may include the hypertension risk stratification 404, which may categorize the individual into a risk group (e.g., high risk versus low risk, tertiles of risk, quintiles of risk) based on the probability or risk score compared to one or more thresholds. As a non-limiting example, a threshold of 0.85 stratifies low risk individuals (e.g., those for which the hypertension probability score 402 is less than 0.85) from high risk individuals (e.g., those for which the hypertension probability score 402 is greater than or equal to 0.85). The hypertension probability score 402 and the hypertension risk stratification 404 may provide clinicians with a comprehensive assessment of the individual's hypertension risk, enabling more informed decision-making for patient care and follow-up.

Optionally, the machine learning model generates auxiliary predictions based on the ECG feature map (block 610). By way of example, the auxiliary predictions 412 may include additional outputs derived from the ECG data, such as the age prediction 406, the sex prediction 408, and/or the medication prediction 410. These auxiliary predictions 412 may provide supplementary information that may be used for clinical decision-making and/or quality control purposes. For example, the age prediction 406, the sex prediction 408, and/or the medication prediction 410 may serve as a cross-check against patient demographic and medical record information. By generating these auxiliary predictions 412 alongside the hypertension risk prediction 116, the machine learning model may offer a more comprehensive analysis of a patient's cardiovascular health based on the ECG data.

In this way, the procedure 600 enables use of a machine learning model that is trained to output an accurate hypertension risk prediction 116 based on ECG data. The electrocardiogram-based deep learning approach for hypertension prediction described herein may be advantageous due to the challenges in accurately diagnosing hypertension using traditional methods. Office blood pressure measurements can be highly variable and may not reflect a patient's true blood pressure due to factors such as anxiety-induced hypertension or masked hypertension. The ability of the machine learning model to analyze ECG data and provide a hypertension risk prediction may assist clinicians in identifying patients with potential masked hypertension or those at risk of developing hypertension, even when office blood pressure readings appear normal. This approach may also reduce the misdiagnosis of hypertension that is related to the anxiety of being in a medical setting, which may cause an individual's blood pressure to temporarily increase. Accordingly, the techniques described herein may enable earlier interventions and more accurate risk stratification. Moreover, the ability of the machine learning model to generate auxiliary predictions such as biological age estimation, sex prediction, and antihypertensive medication use prediction may provide additional context for interpreting the hypertension risk prediction. This holistic assessment may aid clinicians in patient stratification and tailoring management strategies, particularly in cases where office blood pressure measurements alone may be insufficient or misleading. As yet another example, the ability of the machine learning model to process and analyze ECG data for hypertension risk prediction may provide a standardized and objective assessment tool to complement clinical judgment and office blood pressure measurements. This may be especially useful in cases where there is a discrepancy between office blood pressure readings and clinical suspicion of hypertension. The hypertension risk prediction 116, for instance, may serve as an additional data point for clinicians to consider when deciding whether to pursue further evaluation with 24-hour ambulatory blood pressure monitoring or to initiate antihypertensive therapy.

Having described example procedures in accordance with one or more implementations, consider now an example system and device that can be utilized to implement the various techniques described herein.

### Example System and Device

FIG. 7 illustrates an example system generally at 700 that includes an example computing device 702 that is representative of one or more computing systems and/or devices that may implement the various techniques described herein. This is illustrated through inclusion of the computing device 108. The computing device 702 may be, for example, a server of a service provider, a device associated with a client (e.g., a client device), an on-chip system, and/or any other suitable computing device or computing system.

The example computing device 702 as illustrated includes a processing system 704, one or more computer-readable media 706, and one or more I/O interfaces 708 that are communicatively coupled, one to another. Although not shown, the computing device 702 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 704 is representative of functionality to perform one or more operations using hardware. Accordingly, the processing system 704 is illustrated as including hardware elements 710 that may be configured as processors, functional blocks, and so forth. This may include implementation in hardware as an application specific integrated circuit or other logic device formed using one or more semiconductors. The hardware elements 710 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically executable instructions.

The computer-readable media 706 is illustrated as including memory/storage 712. The memory/storage 712 represents memory/storage capacity associated with one or more computer-readable media. The memory/storage 712 may include volatile media (such as random-access memory (RAM)) and/or nonvolatile media (such as read only memory (ROM), Flash memory, optical disks, magnetic disks, and so forth). The memory/storage 712 may include fixed media (e.g., RAM, ROM, a fixed hard drive, and so on) as well as removable media (e.g., flash memory, a removable hard drive, an optical disc, and so forth). The computer-readable media 706 may be configured in a variety of other ways as further described below.

Input/output interface(s) 708 are representative of functionality to allow a user to enter commands and information to computing device 702, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 702 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

For instance, the terms “module,” “functionality,” and “component” may include a hardware and/or software system that operates to perform one or more functions. For example, a module, functionality, or component may include a computer processor, a controller, or another logic-based device that performs operations based on instructions stored on a tangible and non-transitory computer-readable storage medium, such as a computer memory. Alternatively, a module, functionality, or component may include a hard-wired device that performs operations based on hard-wired logic of the device. Various modules, systems, and components shown in the attached figures may represent the hardware that operates based on software or hardwired instructions, the software that directs hardware to perform the operations, or a combination thereof.

An implementation of the described modules and techniques may be stored on or transmitted across some form of computer-readable media. The computer-readable media may include a variety of media that may be accessed by the computing device 702. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media, and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 702, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 710 and computer-readable media 706 are representative of modules, programmable device logic and/or fixed device logic implemented in a hardware form that may be employed in some examples to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 710. The computing device 702 may be configured to implement particular instructions and/or functions corresponding to the software and/or hardware modules. Accordingly, implementation of a module that is executable by the computing device 702 as software may be achieved at least partially in hardware, e.g., through use of computer-readable storage media and/or hardware elements 710 of the processing system 704. The instructions and/or functions may be executable/operable by one or more articles of manufacture (for example, one or more computing devices 702 and/or processing systems 704) to implement techniques, modules, and examples described herein.

The techniques described herein may be supported by various configurations of the computing device 702 and are not limited to the specific examples of the techniques described herein. This functionality may also be implemented all or in part through use of a distributed system, such as over a “cloud” 714 via a platform 716 as described below.

The cloud 714 includes and/or is representative of a platform 716 for resources 718, which are depicted including the computing device 108. The platform 716 abstracts underlying functionality of hardware (e.g., servers) and software resources of the cloud 714. The resources 718 may include applications and/or data that can be utilized while computer processing is executed on servers that are remote from the computing device 702. The resources 718 can also include services provided over the Internet and/or through a subscriber network, such as a cellular or Wi-Fi network.

The platform 716 may abstract resources and functions to connect the computing device 702 with other computing devices. The platform 716 may also serve to abstract scaling of resources to provide a corresponding level of scale to encountered demand for the resources 718 that are implemented via the platform 716. Accordingly, in an interconnected device example, implementation of functionality described herein may be distributed throughout the system 700. For example, the functionality may be implemented in part on the computing device 702 as well as via the platform 716 that abstracts the functionality of the cloud 714.

Having discussed example details of the techniques for electrocardiogram-based deep learning for hypertension prediction, consider now the following examples to illustrate usage of the techniques.

### Example Application

A Digital Biomarker to Diagnose Hypertension and Stratify Cardiovascular Risk from the ECG

Hypertension affects over 1 billion individuals worldwide and is a major modifiable risk factor for cardiovascular disease (CVD). Although measurement of blood pressure (BP) is considered straightforward, many patient- and measurement-related factors can influence BP measurements, contributing to high inter-visit variability in measured BP. Society guidelines for accurate BP measurement are extensive and include requiring patients to rest for five minutes, ensuring the patient has emptied their bladder, avoiding exercise and caffeine prior to measurement, and averaging BP readings over multiple visits, among other recommendations. However, these recommendations can be difficult to rigorously implement in busy clinical settings.

The high variability of office BP measurement complicates the diagnosis and management of hypertension. An estimated 10-15% of patients have masked hypertension, in which office BP is lower than ambulatory BP, with reported prevalence as high as 50% in African Americans. As with sustained hypertension, masked hypertension carries increased risk of CVD. Guidelines recommend 24-hour ambulatory BP monitoring to evaluate masked hypertension, but ambulatory BP monitoring is used infrequently. There is therefore a need for a convenient method of identifying ambulatory hypertension that does not rely on highly dynamic office BP measurements.

Hypertension is associated with changes in cardiac structure and conduction that are reflected in electrocardiographic (ECG) features such as increased QRS voltages, prolonged QT interval, prolonged P wave duration and PR interval, and abnormal repolarization. However, outside of extreme features such as left ventricular hypertrophy, the ECG features of hypertension are subtle and difficult to make use of in routine clinical practice.

According to the techniques described herein, deep learning was used to identify hypertension from the ECG waveform to facilitate screening and diagnosis of hypertension without reliance on dynamic office BP measurements. Additionally, because the adverse effects of hypertension are mediated in part by the effect of hypertension on the myocardium, which may manifest in the ECG, a deep learning-derived digital biomarker for hypertension may stratify the risk of cardiovascular complications of hypertension, including mortality, heart failure (HF), myocardial infarction (MI), stroke, and aortic dissection or rupture.

### Methods

### Study Population

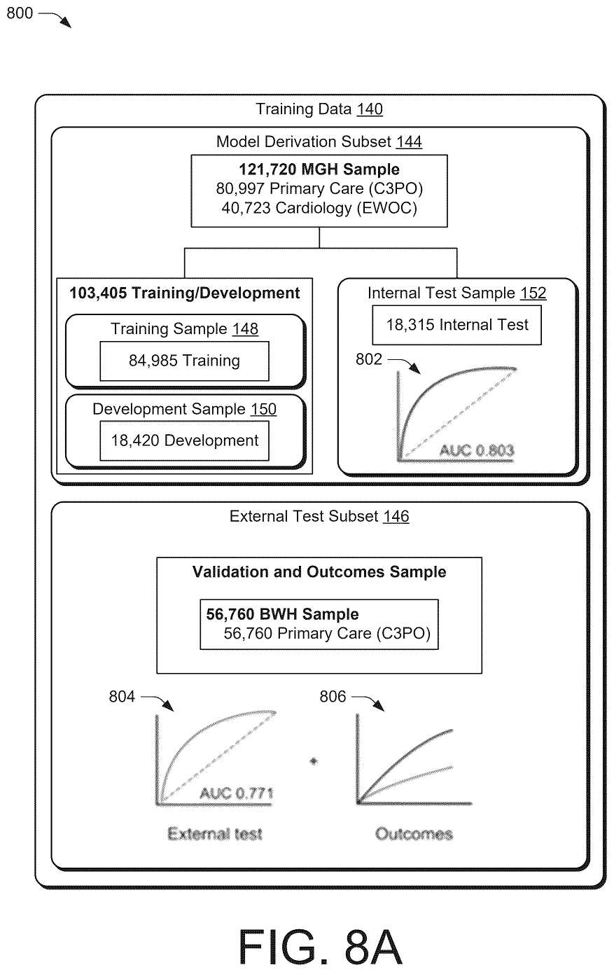

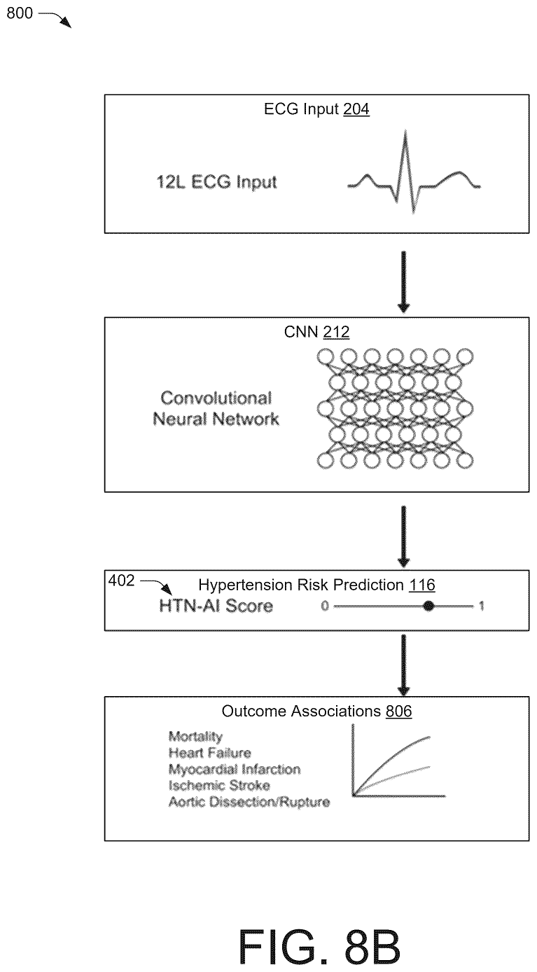

The study samples were derived from two longitudinal electronic health record (EHR)-based cohorts of ambulatory patients within the Mass General Brigham (MGB) healthcare system (FIG. 8A). The first cohort was the Community Care Cohort Project, a cohort of 520,868 patients aged 18 to 89 years with multiple visits to MGB primary care clinics between 2001 and 2018. The cohort was designed to ensure accurate ascertainment of baseline comorbidities by capturing only patients receiving longitudinal primary care within the MGB system and to minimize missingness by using natural language processing to recover missing data from clinical notes. Comparison of the Community Care Cohort Project to a convenience EHR sample demonstrated better calibration for established risk models including the Pooled Cohorts Equation and Cohorts for Heart and Aging Research in Genomic Epidemiology Atrial Fibrillation (CHARGE-AF) risk score. The second cohort was the Enterprise Warehouse of Cardiology, an analogously designed cohort of 99,252 longitudinal cardiology patients between the ages of 18 and 89 years with multiple visits to MGB cardiology clinics between 2000 and 2019. Start of follow up in each cohort was the date of the second clinic visit that determined cohort inclusion.

The model derived in the Example Application, referred to as HTN-AI, was trained on patients in either cohort with one or more 12-lead ECG performed at Massachusetts General Hospital (MGH) within 3 years of the start of follow up. Internal validity was assessed in a hold-out MGH validation sample using the most recent ECG performed within the 3 years before the start of follow up. Patients missing data for age or sex were excluded. External validation and time-to-event analyses were performed in patients in the primary care cohort with 12-lead ECGs done at Brigham and Women's Hospital (BWH) within the 3 years before the start of follow up (BWH external validation sample). Patients with both MGH and BWH ECGs were included only in the training sample, and patients missing data for age, date of last follow up, or sex were excluded.

FIGS. 8A and 8B illustrate a summary 800 of a study overview for the electrocardiogram-based deep learning for hypertension prediction. As shown in FIG. 8A, the training data 140 includes a model derivation subset 144 and an external test subset 146. The model derivation subset 144 comprises a sample of 121,720 individuals from the MGH cohort. This is further divided into a training/development sample having 103,405 individuals and the internal test sample 152 of 18,315 individuals. The training/development sample is subdivided into the training sample 148 of 84,985 individuals and a development sample 150 of 18,420 individuals. The internal test sample 152 is used for internal validation, as represented by an internal validation curve 802. The external test subset 146 includes a validation and outcomes sample of 56,760 individuals from the BWH cohort. The external test subset 146 is used for external validation, represented by the external validation curve 804, and for outcomes analysis, represented by the outcome associations 806.

FIG. 8B depicts how, during training and validation, the ECG input 204, which represents a 12-lead electrocardiogram signal, is input into the CNN 212. The CNN 212 is depicted as a network of interconnected nodes, representing the layers and neurons of the neural network. The output of the CNN 212 is used to generate the hypertension risk prediction 116. The hypertension risk prediction 116 is the hypertension probability score 402 in the Example Application, shown as a scale from 0 to 1, indicating the likelihood of hypertension.

Following the hypertension risk prediction 116, outcome associations 806 are generated. This process includes determining the relationships between the hypertension probability score 402 and various cardiovascular outcomes, including mortality, heart failure, myocardial infarction, ischemic stroke, and aortic dissection/rupture.

### Patient Characteristics and Outcomes

Age, sex, race, ethnicity, comorbidities, and outcomes were determined using EHR data. Race and ethnicity were extracted from a combined EHR field. Baseline comorbidities, including atrial fibrillation, chronic kidney disease, coronary artery disease, diabetes mellitus, HF, and hypertension, were determined by presence of a single International Classification of Diseases (ICD)-9 or ICD-10 code corresponding to the comorbidity by the start of cohort follow up, except for atrial fibrillation and HF. Atrial fibrillation was defined using an algorithm that uses diagnostic and procedural codes and ECG reports. HF was defined using diagnosis codes for a primary diagnosis of HF for inpatient encounters. Hypertension medication use was determined from EHR medication records. Baseline BP was defined as the most recent BP measured within the 3 years prior to start follow up and was obtained using structured EHR data and natural language processing of clinical notes. Within the BWH validation sample, patients who underwent 24-hour ambulatory BP monitoring were identified using Current Procedural Terminology codes (93784, 93786, 93788, 93790, and A4670), and ambulatory BP monitoring results were manually reviewed to obtain 24-hour average systolic and diastolic BP.

CVD outcomes included all-cause mortality, HF, MI, stroke, and aortic dissection or rupture. Dates of death were determined using EHR records. Dates of incident HF were defined as the date of the first inpatient diagnosis code for HF in the primary position. Dates of incidence of other outcomes were the date of the first corresponding diagnosis code after the start of follow up.

### Electrocardiogram Acquisition and Preprocessing

ECGs without abnormalities were identified via text matching using physician interpretations. ECGs were resampled (e.g., via the data preprocessor 132) to 500 Hz and zero-padded to 10 seconds for a resulting model input tensor of shape 5000×12. For example, prior to presentation to the model, voltages for each ECG were normalized to have a mean of zero and standard deviation of one. ECGs recorded at 250 Hz were bilinearly upsampled to 500 Hz, and leads with missing data were zero-padded such that all 12 leads contained 5000 voltages.

### Model Development and Training

Patients were considered to have prevalent hypertension if they had 1) a baseline diagnosis of hypertension by diagnosis code, 2) baseline systolic BP (SBP) ≥140 mmHg, if available, and/or 3) baseline diastolic BP (DBP) ≥90 mmHg, if available. Multiple ECGs were used per patient during training, including ECGs before and after the start of follow up. During validation and time-to-event analyses, only the most recent ECG before the start of follow up was used. Training (e.g., the training sample 148), development (e.g., the development sample 150), and internal validation (e.g., the internal test sample 152) samples were generated using a 70/15/15 percent split at the patient level.

The model includes a series of 1-dimensional convolutions followed by nonlinear activation functions and downsampling operations, which transform the input from a 5000×12 tensor to a 256-dimension embedding (e.g., the ECG vector 234), which is used by output layers (e.g., the one or more dense layers 236) to predict hypertension, antihypertensive medication use, sex, and age. The cross-entropy loss for the categorical tasks and the logarithmic hyperbolic cosine loss for age regression were summed and minimized using the Adam stochastic gradient descent algorithm. The model was implemented with the ML4H Python library (version 0.0.4) using the Tensorflow (version 2.9.1) framework.

Model hyperparameters including depth, width, convolutional kernel size, and activation function were selected through Bayesian hyperparameter optimization. The resulting architecture included 15,072,839 parameters organized into 3 blocks of densely-connected parallel convolutions with kernel size of 71, which passed through the swish activation function and were merged together and down-sampled by max-pooling, as in the Densenet architecture. A dropout rate of 0.5 on dense layers and spatial dropout rate of 0.2 on convolutional layers were used during training to stochastically sample from the space of architectures and mitigate overfitting. Weights were updated using the Adam optimizer with batch size of 24 and an initial learning rate of 5e-4. The learning rate decayed by a factor of 0.5 after 32 epochs without an improvement in validation loss. Early stopping was used to checkpoint the model parameters with minimum validation loss during training, and those model parameters were used for all subsequent evaluation.

### Deep Learning Model Sensitivity Analysis

To better understand what drives the performance of HTN-AI, ablation studies were conducted using different definitions of hypertension and different sets of auxiliary tasks in model training. The primary model defined prevalent hypertension as any combination of 1) a baseline diagnosis of hypertension by ICD-9 or ICD-10 codes, 2) baseline systolic BP ≥140 mmHg, and/or 3) baseline diastolic BP ≥90 mmHg. The primary model was trained using this hypertension label with auxiliary tasks of age regression, sex classification, and classification of baseline antihypertensive medication use. Three other combinations of hypertension definitions and task combinations were tested. The first was a single-task model using the same definition of prevalent hypertension without simultaneous age regression, sex classification, or classification of baseline antihypertensive medication use. The second was a single-task model that also incorporated use of anti-hypertensive medication into the definition of prevalent hypertension. For this model anti-hypertensive medication use was not used as an auxiliary training task. The third was a single-task model that included only ICD-9 or ICD-10 code diagnosis of hypertension without consideration of baseline BP.

For models that incorporated baseline BP measurements, if baseline BP measurements were not available, they were not used in determining the training label. Table 1 details performance of the primary model and sensitivity analysis models in both the MGH and BWH test samples.

TABLE 1
Model sensitivity analyses

| | | | Classification | | Sensitivity | | | | |
|---|---|---|---|---|---|---|---|---|---|
| Model | N | Cutoff | Accuracy | AUROC | Recall | Specificity | AUPRC | Precision | F1 |
| Primary Model | 56,760 | 0 · 85 | 0.566 | 0.771 | 0.329 | 0.917 | 0.811 | 0.855 | 0.475 |
| (Hypertension | | | | | | | | | |
| diagnosis/high BP) - | | | | | | | | | |
| BWH Test | | | | | | | | | |
| Primary Model | 18,315 | 0 · 85 | 0.587 | 0.803 | 0.412 | 0.908 | 0.865 | 0.891 | 0.563 |
| (Hypertension | | | | | | | | | |
| diagnosis/high BP) - | | | | | | | | | |
| MGH Test | | | | | | | | | |
| Hypertension | 56,760 | 0 · 89 | 0.570 | 0.780 | 0.418 | 0.909 | 0.880 | 0.911 | 0.573 |
| diagnosis/high BP/ | | | | | | | | | |
| antihypertensive | | | | | | | | | |
| medication - BWH | | | | | | | | | |
| Test | | | | | | | | | |
| Hypertension | 18,315 | 0 · 89 | 0.618 | 0.821 | 0.509 | 0.910 | 0.920 | 0.938 | 0.660 |
| diagnosis/high BP/ | | | | | | | | | |
| antihypertensive | | | | | | | | | |
| medication - MGH | | | | | | | | | |
| Test | | | | | | | | | |
| Hypertension | 56,760 | 0 · 80 | 0.603 | 0.774 | 0.345 | 0.914 | 0.787 | 0.829 | 0.488 |
| diagnosis - BWH | | | | | | | | | |
| Test | | | | | | | | | |
| Hypertension | 18,315 | 0 · 80 | 0.602 | 0.802 | 0.412 | 0.914 | 0.852 | 0.887 | 0.563 |
| diagnosis - MGH | | | | | | | | | |
| Test | | | | | | | | | |
| Hypertension | 56,760 | 0 · 81 | 0.574 | 0.774 | 0.343 | 0.917 | 0.817 | 0.860 | 0.491 |
| diagnosis/high BP, | | | | | | | | | |
| without co-training | | | | | | | | | |
| tasks - BWH Test | | | | | | | | | |
| Hypertension | 18,315 | 0 · 81 | 0.604 | 0.805 | 0.441 | 0.903 | 0.868 | 0.893 | 0.590 |
| diagnosis/high BP, | | | | | | | | | |
| without co-training | | | | | | | | | |
| tasks - MGH Test | | | | | | | | | |

### Outcome Association Sensitivity Analysis

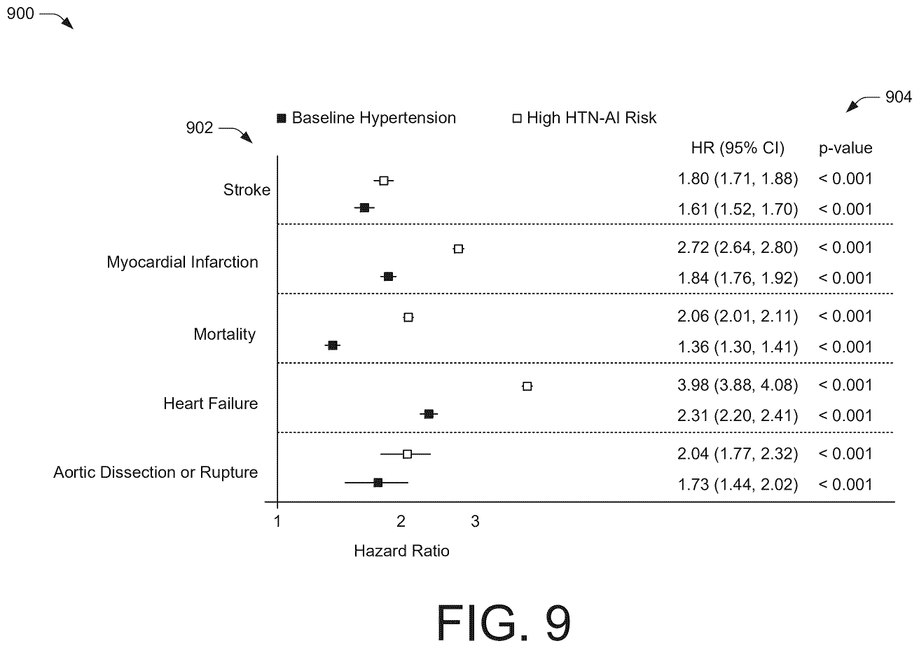

A sensitivity analysis was also performed examining the association of HTN-AI with incident cardiovascular disease in patients with normal ECGs, to assess whether the association is sensitive to the presence of abnormal ECG findings. The text of cardiologist ECG reads was obtained from the MUSE ECG database. The text of cardiologist ECG reads was searched for the phrase “Normal Sinus Rhythm Normal ECG,” which is a common phrase entered using a text macro used by cardiologists in the system for ECGs that contain no abnormalities. This was intended to be a specific rather than sensitive search for ECGs without clinically significant abnormal features. ECGs in the BWH test sample that contained this phrase were considered normal, and this subset of ECGs was used in an identical cause-specific cumulative incidence regression as described below. The results are depicted in FIG. 9.

FIG. 9 depicts an analysis 900 comparing hazard ratios for various cardiovascular outcomes between baseline hypertension and high HTN-AI risk. The analysis 900 represents an age- and sex-adjusted sub-distribution of hazard ratios for high HTN-AI risk and baseline hypertension diagnosis. The analysis 900 includes a forest plot 902 and statistics 904. The forest plot 902 displays hazard ratios and confidence intervals for different cardiovascular outcomes, including stroke, myocardial infarction, mortality, heart failure, and aortic dissection or rupture. Separate markers are shown for baseline hypertension diagnosis (black-filled squares) and high HTN-AI risk (white- or open-filled squares) for each outcome. The statistics 904 provide numerical hazard ratio (HR) values with 95% confidence intervals and p-values for each outcome. The p-values are consistently less than 0.001 for all comparisons, indicating statistical significance across all outcomes.

### Statistical Analyses

Normally distributed data are reported as means (standard deviation [SD]), non-normally distributed data as median (interquartile range) and categorical data as counts (%). High and low HTN-AI risk groups were determined using the HTN-AI score cutoff that achieves 90% specificity in the model development sample. Differences in baseline comorbidities between high and low HTN-AI risk groups were tested using the chi-square test. Model performance for hypertension classification is reported as area under the receiver-operator curve (AUROC), sensitivity/recall, specificity, precision, average precision, and F1 score.

The association between HTN-AI risk group and elevated 24-hour ambulatory BP was tested using logistic regression with adjustment for age, sex, and use of an antihypertensive medication prior to ambulatory BP monitoring, and 2-sided Wald p-values are reported. For incident disease analyses, cause-specific cumulative incidence curves for each outcome are shown with 2-sided p-values for equivalence between groups calculated by Gray's test, accounting for competing risk of death for outcomes other than mortality. Cause-specific Fine-Gray regression was performed, accounting for competing risk of death for outcomes other than mortality, with adjustment for age and sex. Subdistribution hazard ratios (HRs) and 95% confidence intervals with 2-sided p-values are reported. Patients were right-censored at the last EHR encounter or on 8/31/19. Patients with missing BP data (n=3,349 [5.9%]) were excluded to facilitate comparison between models using HTN-AI score, SBP, and pulse pressure.

Statistical analyses were performed using R (version 4.2.1) with packages cmprsk (version 2.2-11), survival (version 3.4-0), survminer (version 0.4.9), and yardstick (version 1.1.0).

### Results

### Population Characteristics

FIGS. 8A and 8B depict an overview of the study. The MGH sample comprised 121,720 patients, who were divided into training, development, and internal validation samples (Table 2). There were 752,415 ECGs in the training and development samples, with median 3 (1-6) ECGs per patient and a median time difference between ECG and start of follow up of −121 (−561 to 409) days. Mean age in the MGH sample was 57.3 (16.8) years and 76,499 (62.8%) patients had known hypertension. Among those with BP measurements (n=36,718 [30.2%] missing SBP; n=36,725 [30.2%] missing DBP), 18,835 (22.2%) had SBP ≥140 mmHg, and 8,680 (10.2%) had SBP ≥90 mmHg. As expected, among the 79,751 (65.5%) patients with baseline hypertension or elevated baseline BP, cardiac comorbidities and risk factors were enriched, including hyperlipidemia (57,678 [72.3%] vs 14,233 [33.9%]) and coronary artery disease (36,076 [45.2%] vs 5,707 [13.6%]; Table 3). Baseline features were similar between splits of the MGH sample (Table 4).

TABLE 2
Patient characteristics

| | Massachusetts General Hospital | | Brigham and Women's Hospital | |
|---|---|---|---|---|
| | Model Development | | External Validation and Outcomes | |
| N, N | 121, 720 | | 56, 760 | |
| Age (y), mean ± standard deviation | 57.3 ± 16.8 | | 55.1 ± 16.2 | |
| Sex, n (%) | | | | |
| Female | 58,970 | (48.4) | 33,755 | (59.5) |
| Male | 62,750 | (51.6) | 23,005 | (40.5) |
| Race or Ethnicity, n (%) | | | | |
| Asian or Pacific Islander | 4,481 | (3.7) | 1,488 | (2.6) |
| Black | 6,523 | (5.4) | 7,407 | (13.0) |
| Hispanic or Latino | 4,850 | (4.0) | 4,494 | (7.9) |
| Multiple/Other | 3,989 | (3.3) | 2,149 | (3.8) |
| Unknown | 3,299 | (2.7) | 2,609 | (4.6) |
| White | 98,578 | (81.0) | 38,613 | (68.0) |
| Atrial Fibrillation, n (%) | 21,307 | (17.5) | 5,588 | (9.8) |
| Chronic Kidney Disease, n (%) | 14,346 | (11.8) | 5,824 | (10.3) |
| Coronary Artery Disease, n (%) | 41,783 | (34.3) | 15,320 | (27.0) |
| Diabetes Mellitus, n (%) | 23,679 | (19.5) | 9,750 | (17.2) |
| Heart Failure, n (%) | 7,124 | (5.9) | 2,305 | (4.1) |
| Hyperlipidemia, n (%) | 71,911 | (59.1) | 25,679 | (45.2) |
| Hypertension, n (%)^{a} | 76,499 | (62.8) | 31,014 | (54.6) |
| Stroke, n (%) | 7,948 | (6.5) | 1,957 | (3.4) |
| Myocardial Infarction, n (%) | 17,198 | (14.1) | 6,892 | (12.1) |
| Antihypertensive Medication Use, n | 68,751 | (56.5) | 31,154 | (54.9) |
| (%) | | | | |
| Systolic Blood Pressure (mmHg) | | | | |
| Mean ± standard deviation | 126.6 ± 17.7 | | 127.6 ± 18.0 | |
| Unknown | 36,718 | (30.2) | 3,339 | (5.9) |
| Systolic Blood Pressure ≥140 | 18,835 | (22.2) | 12,494 | (23.4) |
| mmHg, n (%) | | | | |
| Diastolic Blood Pressure (mmHg) | | | | |
| Mean ± standard deviation | 75.5 ± 10.6 | | 75.8 ± 11.2 | |
| Unknown | 36,725 | (30.2) | 3,340 | (5.9) |
| Diastolic Blood Pressure ≥90 mmHg, | 8,680 | (10.2) | 6,240 | (11.7) |
| n (%) | | | | |
| Hypertension Diagnosis or High | 79,751 | (65.5) | 33,906 | (59.7) |
| Baseline Blood Pressure, n (%) | | | | |
| Source Population, n (%) | | | | |
| Cardiology Cohort | 40,723 | (33.5) | 0 | (0.0) |
| Primary Care Cohort | 80,997 | (66.5) | 56,760 | (100.0) |

^{a}By ICD-9 or ICD-10 code

TABLE 3
Population characteristics by training label

| | MGH Sample (Model Development) | | | | BWH Sample(External Validation and | | | |
|---|---|---|---|---|---|---|---|---|
| | N = 121720 | | | | Outcomes) N = 56760 | | | |
| | Negative | | Positive | | Negative | | Positive | |
| | Hypertension | | Hypertension | | Hypertension | | Hypertension | |
| | Label | | Label | | Label | | Label | |
| N, N | 41, 969 | | 79, 751 | | 22, 854 | | 33, 906 | |
| Age (y), Mean ± SD | 46.7 ± 15.9 | | 62.8 ± 14.5 | | 47.1 ± 15.3 | | 60.5 ± 14.4 | |
| Sex, n (%) | | | | | | | | |
| Female | 23,270 | (55.4) | 35,700 | (44.8) | 14,874 | (65.1) | 18,881 | (55.7) |
| Male | 18,699 | (44.6) | 44,051 | (55.2) | 7,980 | (34.9) | 15,025 | (44.3) |
| Race or Ethnicity, n (%) | | | | | | | | |
| Asian or Pacific | 1,951 | (4.6) | 2,530 | (3.2) | 741 | (3.2) | 747 | (2.2) |
| Islander | | | | | | | | |
| Black | 2,113 | (5.0) | 4,410 | (5.5) | 2,365 | (10.3) | 5,042 | (14.9) |
| Hispanic or Latino | 2,272 | (5.4) | 2,578 | (3.2) | 1,844 | (8.1) | 2,650 | (7.8) |
| Multiple/Other | 1,814 | (4.3) | 2,175 | (2.7) | 934 | (4.1) | 1,215 | (3.6) |
| Unknown | 1,133 | (2.7) | 2,166 | (2.7) | 966 | (4.2) | 1,643 | (4.8) |
| White | 32,686 | (77.9) | 65,892 | (82.6) | 16,004 | (70.0) | 22,609 | (66.7) |
| Atrial Fibrillation, n (%) | 3,578 | (8.5) | 17,729 | (22.2) | 1,257 | (5.5) | 4,331 | (12.8) |
| Chronic Kidney Disease, | 1,246 | (3.0) | 13,100 | (16.4) | 736 | (3.2) | 5,088 | (15.0) |
| n (%) | | | | | | | | |
| Coronary Artery Disease, | 5,707 | (13.6) | 36,076 | (45.2) | 3,484 | (15.2) | 11,836 | (34.9) |
| n (%) | | | | | | | | |
| Diabetes Mellitus, n (%) | 2,564 | (6.1) | 21,115 | (26.5) | 1,344 | (5.9) | 8,406 | (24.8) |
| Heart Failure, n (%) | 614 | (1.5) | 6,510 | (8.2) | 372 | (1.6) | 1,933 | (5.7) |
| Hyperlipidemia, n (%) | 14,233 | (33.9) | 57,678 | (72.3) | 6,087 | (26.6) | 19,592 | (57.8) |
| Hypertension, n (%)¹ | 0 | (0.0) | 76,499 | (95.9) | 0 | (0.0) | 31,014 | (91.5) |
| Stroke, n (%) | 1,096 | (2.6) | 6,852 | (8.6) | 269 | (1.2) | 1,688 | (5.0) |
| Myocardial Infarction, n | 1,519 | (3.6) | 15,679 | (19.7) | 1,179 | (5.2) | 5,713 | (16.8) |
| (%) | | | | | | | | |
| Antihypertensive | 9,497 | (22.6) | 59,254 | (74.3) | 5,148 | (22.5) | 26,006 | (76.7) |
| Medication Use, n (%) | | | | | | | | |
| Systolic Blood Pressure | | | | | | | | |
| (mmHg) | | | | | | | | |
| Mean ± SD | 116.1 ± 11.1 | | 132.2 ± 18.0 | | 116.8 ± 11.1 | | 134.7 ± 18.1 | |
| Unknown | 12,429 | (29.6) | 24,289 | (30.5) | 1,570 | (6.9) | 1,769 | (5.2) |
| Systolic Blood | 0 | (0.0) | 18,835 | (34.0) | 0 | (0.0) | 12,494 | (38.9) |
| Pressure ≥140 mmHg, n (%) | | | | | | | | |
| Diastolic Blood Pressure | | | | | | | | |
| (mmHg) | | | | | | | | |
| Mean ± SD | 72.1 ± 8.1 | | 77.2 ± 11.3 | | 71.6 ± 8.6 | | 78.6 ± 11.8 | |
| Unknown | 12,430 | (29.6) | 24,295 | (30.5) | 1,568 | (6.9) | 1,772 | (5.2) |
| Diastolic Blood | 0 | (0.0) | 8,680 | (15.7) | 0 | (0.0) | 6,240 | (19.4) |
| Pressure ≥90 mmHg, n (%) | | | | | | | | |
| Source Population, n (%) | | | | | | | | |
| Cardiology Cohort | 8,949 | (21.3) | 31,774 | (39.8) | | | | |
| Primary Care Cohort | 33,020 | (78.7) | 47,977 | (60.2) | 22,854 | (100.0) | 33,906 | (100.0) |

¹By ICD-9 or ICD-10 code

TABLE 4
Population characteristics by subset of the training sample

| | MGH Training | | MGH Development | | MGH Test | |
|---|---|---|---|---|---|---|
| | Sample | | Sample | | Sample | |
| N, N | 84, 985 | | 18, 420 | | 18, 315 | |
| Age (y), Mean ± SD | 57.3 ± 16.8 | | 57.4 ± 16.9 | | 57.1 ± 16.9 | |
| Sex, n (%) | | | | | | |
| Female | 41,125 | (48.4) | 8,886 | (48.2) | 8,959 | (48.9) |
| Male | 43,860 | (51.6) | 9,534 | (51.8) | 9,356 | (51.1) |
| Race or Ethnicity, n (%) | | | | | | |
| Asian or Pacific Islander | 3,203 | (3.8) | 653 | (3.5) | 625 | (3.4) |
| Black | 4,570 | (5.4) | 991 | (5.4) | 962 | (5.3) |
| Hispanic or Latino | 3,396 | (4.0) | 713 | (3.9) | 741 | (4.0) |
| Multiple/Other | 2,799 | (3.3) | 609 | (3.3) | 581 | (3.2) |
| Unknown | 2,303 | (2.7) | 511 | (2.8) | 485 | (2.6) |
| White | 68,714 | (80.9) | 14,943 | (81.1) | 14,921 | (81.5) |
| Atrial Fibrillation, n (%) | 14,898 | (17.5) | 3,237 | (17.6) | 3,172 | (17.3) |
| Chronic Kidney Disease, n (%) | 10,098 | (11.9) | 2,121 | (11.5) | 2,127 | (11.6) |
| Coronary Artery Disease, n (%) | 29,155 | (34.3) | 6,389 | (34.7) | 6,239 | (34.1) |
| Diabetes Mellitus, n (%) | 16,581 | (19.5) | 3,591 | (19.5) | 3,507 | (19.1) |
| Heart Failure, n (%) | 4,984 | (5.9) | 1,077 | (5.8) | 1,063 | (5.8) |
| Hyperlipidemia, n (%) | 50,345 | (59.2) | 10,899 | (59.2) | 10,667 | (58.2) |
| Hypertension, n (%)¹ | 53,578 | (63.0) | 11,529 | (62.6) | 11,392 | (62.2) |
| Stroke, n (%) | 5,578 | (6.6) | 1,239 | (6.7) | 1,131 | (6.2) |
| Myocardial Infarction, n (%) | 11,986 | (14.1) | 2,650 | (14.4) | 2,562 | (14.0) |
| Antihypertensive Medication Use, n (%) | 48,014 | (56.5) | 10,449 | (56.7) | 10,288 | (56.2) |
| Systolic Blood Pressure (mmHg) | | | | | | |
| Mean ± SD | 126.6 ± 17.7 | | 126.5 ± 17.6 | | 126.5 ± 17.8 | |
| Unknown | 25,632 | (30.2) | 5,591 | (30.4) | 5,495 | (30.0) |
| Systolic Blood Pressure ≥140 mmHg, n (%) | 13,145 | (22.1) | 2,815 | (21.9) | 2,875 | (22.4) |
| Diastolic Blood Pressure (mmHg) | | | | | | |
| Mean ± SD | 75.5 ± 10.6 | | 75.4 ± 10.6 | | 75.5 ± 10.6 | |
| Unknown | 25,634 | (30.2) | 5,595 | (30.4) | 5,496 | (30.0) |
| Diastolic Blood Pressure ≥90 mmHg, n (%) | 6,040 | (10.2) | 1,336 | (10.4) | 1,304 | (10.2) |
| Hypertension Diagnosis or High Baseline Blood | 55,852 | (65.7) | 12,044 | (65.4) | 11,855 | (64.7) |
| Pressure, n (%) | | | | | | |
| Source Population, n (%) | | | | | | |
| Cardiology Cohort | 28,506 | (33.5) | 6,215 | (33.7) | 6,002 | (32.8) |
| Primary Care Cohort | 56,479 | (66.5) | 12,205 | (66.3) | 12,313 | (67.2) |

¹By ICD-9 or ICD-10 code

The BWH external validation and outcomes sample included 56,760 patients with mean age 55.1 (16.2) years. The prevalence of hypertension (31,014 [54.6%]) was lower, though the rates of elevated SBP (12,494 [23.4%]; n=3,339 [5.9%] missing) and DBP (6,240 [11.7%]; n=3,340 [5.9%] missing) were similar. Only the closest ECG before the start of follow up was used during external validation, with a median time difference of −533 (−723 to −279) days. As in the MGH sample, cardiac comorbidities and risk factors were enriched among the 33,906 (59.7%) patients with hypertension or elevated baseline BP (Table 3).

### Model Performance

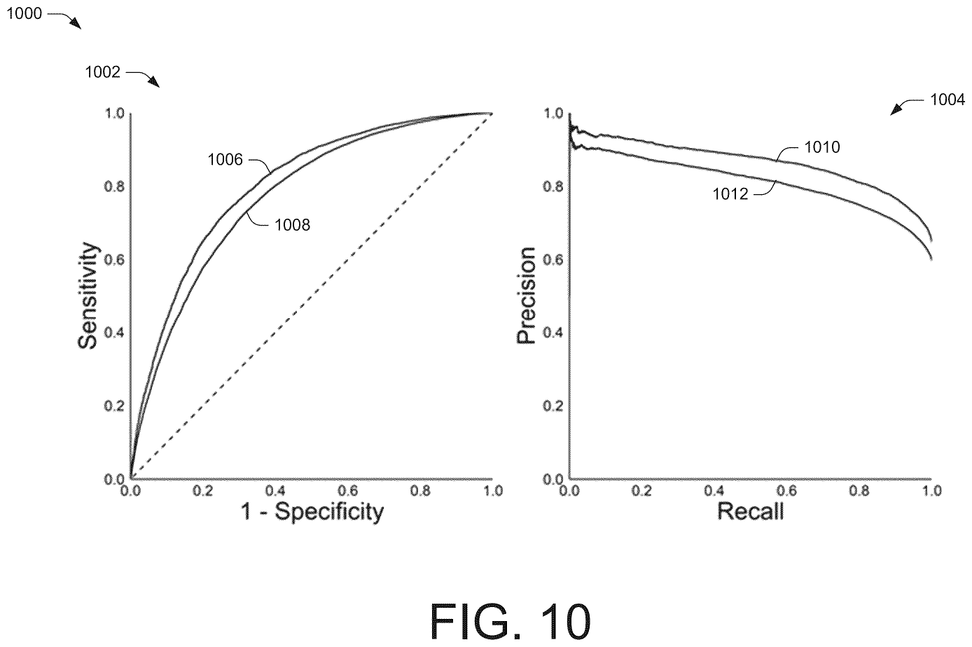

The HTN-AI score discriminated patients with baseline hypertension or elevated BP with an AUROC of 0.803 (0.796-0.810) in the MGH internal validation sample and 0.771 (0.767-0.775) in the BWH validation sample (FIG. 10). Average precision was 0.865 (0.858-0.872) in the MGH internal validation sample and 0.811 (0.806-0.816) in the BWH external validation sample. Other performance metrics and model sensitivity analyses are shown in Table 1.

FIG. 10 depicts an analysis 1000 of model performance in the internal and external test sets for an electrocardiogram-based deep learning model for hypertension prediction. The analysis 1000 includes a first graph 1002 and a second graph 1004. The first graph 1002 shows receiver operating characteristic (ROC) curves, plotting sensitivity (vertical axis) against 1—specificity (horizontal axis). A diagonal dashed line in the first graph 1002 represents a baseline random classifier performance. The second graph 1004 shows precision-recall curves, plotting precision (vertical axis) against recall (horizontal axis). In the first graph 1002, a first ROC 1006 corresponds to the MGH test sample, and a second ROC 1008 corresponds to the BWH test sample. In the second graph 1004, a first precision-recall curve 1010 corresponds to the MGH test sample, and a second precision-recall curve 1012 corresponds to the BWH test sample.

Associations with Cardiovascular Disease

In total, 13,065 (23.0%) patients in the BWH sample had high HTN-AI risk, using a HTN-AI score threshold of 0.85 defined to achieve 90% specificity in the MGH development sample. Baseline comorbidities were enriched among high HTN-AI risk patients with the greatest relative differences for baseline hypertension (10,719 [82.0%] versus 20,293 [46.4%], p<0.001) and coronary artery disease (6,794 [52.0%] versus 8,526 [19.5%], p<0.001; Table 5).

TABLE 5
Population characteristics by HTN-AI risk group

| | MGH Sample (Model | | | | | BWH Sample (External Validation | | | | |
|---|---|---|---|---|---|---|---|---|---|---|
| | Development) N = 121720 | | | | | and Outcomes) N = 56760 | | | | |
| | Low HTN- | | High | | p- | Low HTN- | | High | | p- |
| | AI | | HTN-AI | | value¹ | AI | | HTN-AI | | value¹ |
| N, N | 85, 132 | | 36, 588 | | | 43, 695 | | 13, 065 | | |
| Age (y), Mean ± SD | 51.7 ± 15.6 | | 70.3 ± 11.5 | | <0.001 | 51.1 ± 15.0 | | 68.5 ± 12.3 | | <0.001 |
| Sex, n (%) | | | | | <0.001 | | | | | <0.001 |
| Female | 43,702 | (51.3) | 15,268 | (41.7) | | 27,051 | (61.9) | 6,704 | (51.3) | |
| Male | 41,430 | (48.7) | 21,320 | (58.3) | | 16,644 | (38.1) | 6,361 | (48.7) | |
| Race or Ethnicity, n (%) | | | | | <0.001 | | | | | <0.001 |
| Asian or Pacific Islander | 3,625 | (4.3) | 856 | (2.3) | | 1,307 | (3.0) | 181 | (1.4) | |
| Black | 5,157 | (6.1) | 1,366 | (3.7) | | 5,806 | (13.3) | 1,601 | (12.3) | |
| Hispanic or Latino | 4,011 | (4.7) | 839 | (2.3) | | 3,699 | (8.5) | 795 | (6.1) | |
| Multiple/Other | 3,271 | (3.8) | 718 | (2.0) | | 1,809 | (4.1) | 340 | (2.6) | |
| Unknown | 2,210 | (2.6) | 1,089 | (3.0) | | 1,875 | (4.3) | 734 | (5.6) | |
| White | 66,858 | (78.5) | 31,720 | (86.7) | | 29,199 | (66.8) | 9,414 | (72.1) | |
| Atrial Fibrillation, n (%) | 7,659 | (9.0) | 13,648 | (37.3) | <0.001 | 2,234 | (5.1) | 3,354 | (25.7) | <0.001 |
| Chronic Kidney Disease, n (%) | 5,875 | (6.9) | 8,471 | (23.2) | <0.001 | 3,153 | (7.2) | 2,671 | (20.4) | <0.001 |
| Coronary Artery Disease, n (%) | 19,374 | (22.8) | 22,409 | (61.2) | <0.001 | 8,526 | (19.5) | 6,794 | (52.0) | <0.001 |
| Diabetes Mellitus, n (%) | 11,463 | (13.5) | 12,216 | (33.4) | <0.001 | 5,582 | (12.8) | 4,168 | (31.9) | <0.001 |
| Heart Failure, n (%) | 1,863 | (2.2) | 5,261 | (14.4) | <0.001 | 703 | (1.6) | 1,602 | (12.3) | <0.001 |
| Hyperlipidemia, n (%) | 43,693 | (51.3) | 28,218 | (77.1) | <0.001 | 17,534 | (40.1) | 8,145 | (62.3) | <0.001 |
| Hypertension, n (%)² | 43,905 | (51.6) | 32,594 | (89.1) | <0.001 | 20,295 | (46.4) | 10,719 | (82.0) | <0.001 |
| Stroke, n (%) | 3,459 | (4.1) | 4,489 | (12.3) | <0.001 | 961 | (2.2) | 996 | (7.6) | <0.001 |
| Myocardial Infarction, n (%) | 6,586 | (7.7) | 10,612 | (29.0) | <0.001 | 3,187 | (7.3) | 3,705 | (28.4) | <0.001 |
| Antihypertensive Med Use, n (%) | 37,943 | (44.6) | 30,808 | (84.2) | <0.001 | 20,316 | (46.5) | 10,838 | (83.0) | <0.001 |
| Systolic Blood Pressure (mmHg) | | | | | <0.001 | | | | | <0.001 |
| Mean ± SD | 124.5 ± 16.7 | | 132.1 ± 19.1 | | | 125.6 ± 16.8 | | 134.6 ± 20.0 | | |
| Unknown | 23,659 | (27.8) | 13,059 | (35.7) | | 2,106 | (4.8) | 1,233 | (9.4) | |
| SBP ≥140 mmHg, n (%) | 11,096 | (18.1) | 7,739 | (32.9) | <0.001 | 8,098 | (19.5) | 4,396 | (37.2) | <0.001 |
| Diastolic Blood Pressure (mmHg) | | | | | <0.001 | | | | | 0.015 |
| Mean ± SD | 75.8 ± 10.3 | | 74.6 ± 11.3 | | | 75.9 ± 10.9 | | 75.6 ± 12.0 | | |
| Unknown | 23,665 | (27.8) | 13,060 | (35.7) | | 2,106 | (4.8) | 1,234 | (9.4) | |
| DPB ≥90 mmHg, n (%) | 6,371 | (10.4) | 2,309 | (9.8) | 0.018 | 4,732 | (11.4) | 1,508 | (12.7) | <0.001 |
| Training Label, n (%) | | | | | <0.001 | | | | | <0.001 |
| Negative Hypertension Label | 38,433 | (45.1) | 3,536 | (9.7) | | 20,954 | (48.0) | 1,900 | (14.5) | |
| Positive Hypertension Label | 46,699 | (54.9) | 33,052 | (90.3) | | 22,741 | (52.0) | 11,165 | (85.5) | |
| Source Population, n (%) | | | | | <0.001 | | | | | |
| Cardiology Cohort | 20,900 | (24.6) | 19,823 | (54.2) | | | | | | |
| Primary Care Cohort | 64,232 | (75.4) | 16,765 | (45.8) | | 43,695 | (100.0) | 13,065 | (100.0) | |

¹Wilcoxon rank sum test; Pearson's Chi-squared test
²By ICD-9 or ICD-10 code

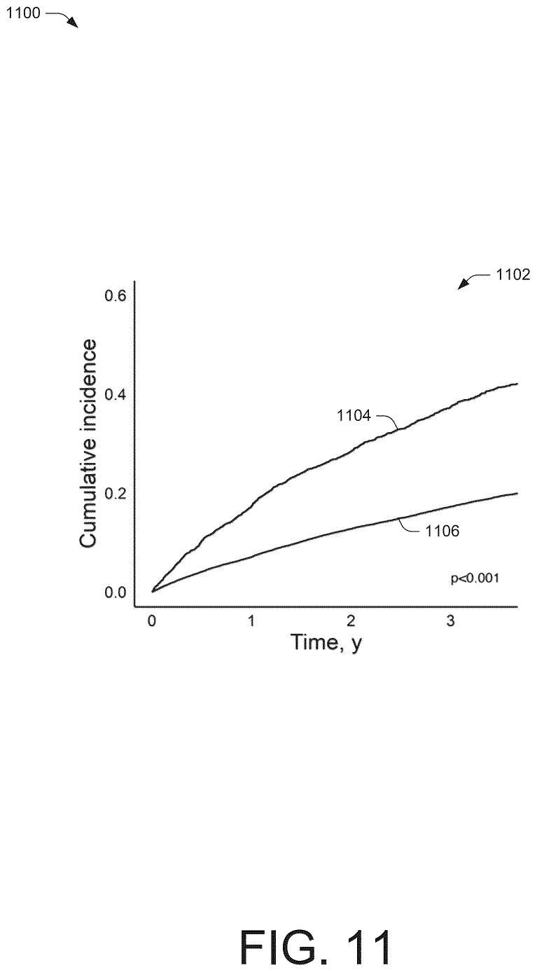

HTN-AI risk was significantly associated with incident hypertension diagnosis (FIG. 11 and Table 6). Among 2,282 patients in the high-risk HTN-AI group there were 1,261 incident hypertension diagnoses (estimated 1-year cumulative incidence of 17.3% [15.7-18.9]) compared to 7,351 diagnoses among 23,043 patients in the HTN-AI low-risk group (estimated 1-year cumulative incidence of 7.1% [6.8-7.4]; p<0.001).

TABLE 6
Cumulative incidence of hypertension stratified by HTN-AI risk

| | | N | 1-Year Cumulative | 3-Year Cumulative | 5-Year Cumulative | p- |
|---|---|---|---|---|---|---|
| | N | Event | Incidence | Incidence | Incidence | value¹ |
| Hypertension | | | | | | <0.001 |
| Low HTN- | 23,043 | 7,351 | 7.1% | 17.2% | 25.1% | |
| AI | | | (6.8%, 7.4%) | (16.7%, 17.8%) | (24.5%, 25.8%) | |
| High | 2,282 | 1,261 | 17.3% | 37.4% | 50.0% | |
| HTN-AI | | | (15.7%, 18.9%) | (35.3%, 39.5%) | (47.7%, 52.3%) | |

¹Gray's Test

FIG. 11 depicts an analysis 1100 of cumulative incidence of hypertension over time based on hypertension risk prediction. The analysis 1100 includes a cumulative incidence graph 1102 that plots cumulative incidence on the y-axis against time in years on the x-axis. The cumulative incidence graph 1102 stratifies patients based on the HTN-AI score, showing a high HTN-AI curve 1104 and a low HTN-AI curve 1106. The high HTN-AI curve 1104 shows a steeper increase in cumulative incidence over time compared to the low HTN-AI curve 1106. The cumulative incidence graph 1102 indicates a p-value of <0.001, suggesting a statistically significant difference between the high HTN-AI curve 1104 and the low HTN-AI curve 1106.

The association between HTN-AI risk and elevated 24-hour ambulatory BP (i.e., average systolic BP ≥125 mmHg or average diastolic BP ≥75 mm Hg) was examined in patients in the BWH sample who had both ambulatory BP monitoring performed for clinical indications and a 12-lead ECG <1 year apart (n=243 total, n=177 [72.8%] with elevated average BP). High HTN-AI risk was associated with an odds ratio of 3.03 (1.47-6.64) for elevated 24-hour ambulatory BP after adjustment for age, sex, and antihypertensive medication use (p=0.004).

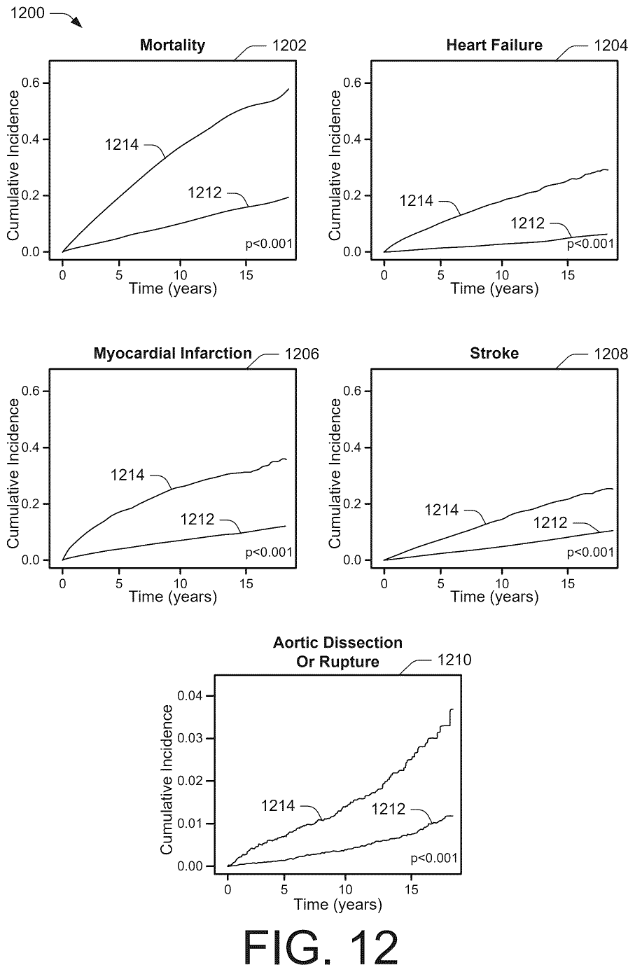

The association of the HTN-AI score with the risk of developing five sequelae of hypertension was further investigated: all-cause mortality, HF, MI, stroke, and aortic dissection or rupture. HTN-AI risk stratified incidence of all examined outcomes (FIG. 12; p<0.001 for all comparisons). Among 12,945 patients with high HTN-AI risk, 4,181 died over median follow up of 5.6 years (estimated 10-year cumulative incidence 21.0% [20.2-21.7]). In comparison, among 43,225 patients at low HTN-AI risk, 4,125 patients died over median follow up of 7.8 years (10-year cumulative incidence 5.4% [5.2-5.6]; p<0.001). Event rates for other outcomes are shown in Table 7.

TABLE 7
Cumulative incidence of cardiovascular outcomes stratified by HTN-AI risk

| | | | 5-Year | 10-Year | 15-Year | 18.5-Year | |
|---|---|---|---|---|---|---|---|
| | | N | Cumulative | Cumulative | Cumulative | Cumulative | p- |
| | N | Event | Incidence | Incidence | Incidence | Incidence | value¹ |
| Mortality | | | | | | | <0.001 |
| Low HTN- | 43,225 | 4,125 | 5.4% (5.2%, | 10.5% (10.1%, | 15.9% (15.4%, | 19.5% (18.7%, | |
| AI | | | 5.6%) | 10.8%) | 16.5%) | 20.2%) | |
| High HTN- | 12,945 | 4,181 | 21.0% (20.2%, | 38.4% (37.4%, | 51.4% (50.0%, | 57.9% (56.0%, | |
| AI | | | 21.7%) | 39.5%) | 52.7%) | 59.8%) | |
| Heart Failure | | | | | | | <0.001 |
| Low HTN- | 42,461 | 1,121 | 1.5% (1.4%, | 2.9% (2.7%, | 4.8% (4.4%, | 6.3% (5.8%, | |
| AI | | | 1.6%) | 3.1%) | 5.1%) | 6.8%) | |
| High HTN- | 11,268 | 1,543 | 10.5% (9.9%, | 18.5% (17.5%, | 25.1% (23.7%, | 29.5% (27.6%, | |
| AI | | | 11.2%) | 19.5%) | 26.4%) | 31.4%) | |
| Myocardial | | | | | | | <0.001 |
| Infarction | | | | | | | |
| Low HTN- | 40,012 | 2,407 | 4.2% (4.0%, | 7.2% (6.9%, | 9.7% (9.3%, | 12.0% (11.4%, | |
| AI | | | 4.5%) | 7.5%) | 10.2%) | 12.7%) | |
| High HTN- | 9,212 | 1,761 | 17.5% (16.6%, | 26.3% (25.1%, | 31.4% (29.9%, | 36.0% (33.7%, | |
| AI | | | 18.4%) | 27.5%) | 32.9%) | 38.3%) | |
| Stroke | | | | | | | <0.001 |
| Low HTN- | 42,197 | 1,821 | 2.2% (2.1%, | 4.7% (4.5%, | 7.9% (7.5%, | 10.4% (9.8%, | |
| AI | | | 2.4%) | 5.0%) | 8.3%) | 11.1%) | |
| High HTN- | 11,855 | 1,282 | 7.7% (7.2%, | 15.3% (14.4%, | 21.4% (20.2%, | 25.0% (23.3%, | |
| AI | | | 8.3%) | 16.2%) | 22.7%) | 26.7%) | |
| Aortic | | | | | | | <0.001 |
| Dissection or | | | | | | | |
| Rupture | | | | | | | |
| Low HTN- | 43,080 | 175 | 0.2% (0.1%, | 0.4% (0.3%, | 0.8% (0.6%, | 1.2% (1.0%, | |
| AI | | | 0.2%) | 0.5%) | 0.9%) | 1.4%) | |
| High HTN- | 12,738 | 144 | 0.8% (0.6%, | 1.4% (1.2%, | 2.5% (2.0%, | 3.7% (2.7%, | |
| AI | | | 1.0%) | 1.8%) | 3.1%) | 4.9%) | |

¹Gray's Test

FIG. 12 depicts a set of graphs 1200 showing cumulative incidence of different cardiovascular outcomes over time, as stratified by HTN-AI risk. The set of graphs 1200 includes a first graph 1202, a second graph 1204, a third graph 1206, a fourth graph 1208, and a fifth graph 1210. The first graph 1202 represents mortality, the second graph 1204 represents heart failure, the third graph 1206 represents myocardial infarction, the fourth graph 1208 represents stroke, and the fifth graph 1210 represents aortic dissection or rupture. Each graph in the set of graphs 1200 includes a low HTN-AI plot 1212 and a high HTN-AI plot 1214. The x-axis for all graphs shows Time in years, while the y-axis shows cumulative incidence. The graphs consistently demonstrate a higher cumulative incidence for the high HTN-AI plot 1214 compared to the low HTN-AI plot 1212 across all outcomes. Each graph in the set of graphs 1200 includes a p-value of <0.001, indicating statistical significance between the low HTN-AI plot 1212 and the high HTN-AI plot 1214.

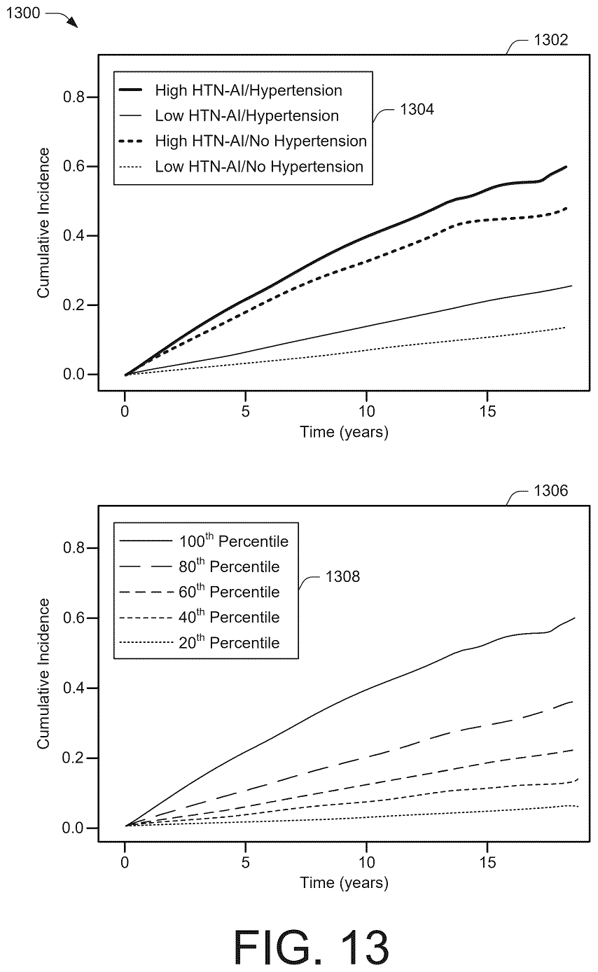

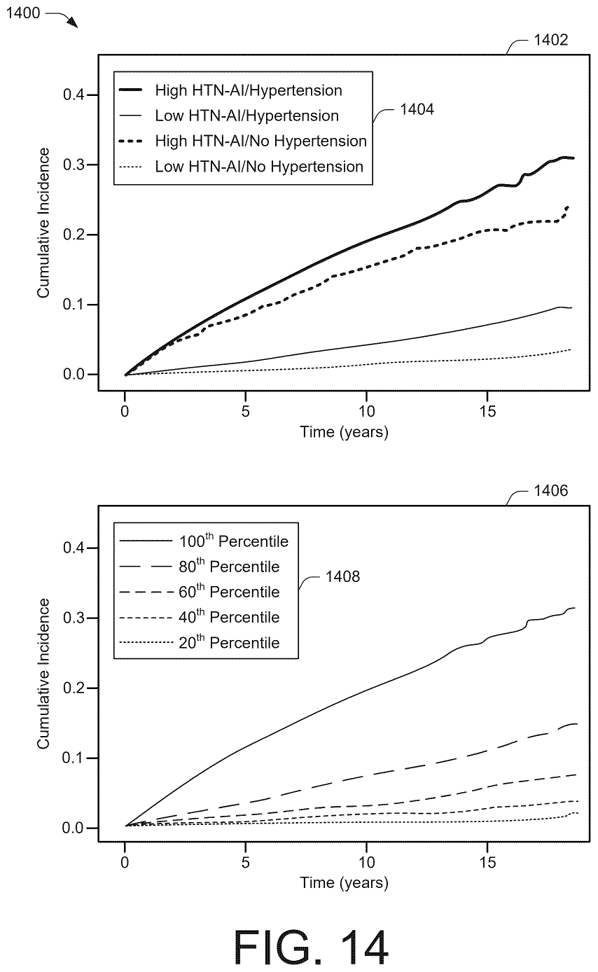

To understand whether the HTN-AI score provides additional information about cardiovascular risk in patients with known hypertension, CVD incidence was examined stratified by both HTN-AI risk and baseline hypertension status. HTN-AI risk stratified cumulative incidence of all-cause mortality among both patients with and without a baseline diagnosis of hypertension (FIG. 13 and Table 7; p<0.001 for equality between all groups). Findings were similar for other outcomes (FIGS. 14-17 and Table 7; p<0.001 for equality between groups across each outcome). Cumulative incidence was further stratified by the quintile of HTN-AI score, and it was found that events were concentrated among patients in the top quintile with a gradient of risk at lower quintiles (FIG. 13, FIGS. 14-17, and Table 7; p <0.001 for equality between quintiles across each outcome).

FIG. 13 depicts an analysis 1300 of a cumulative incidence of all-cause mortality, as stratified by HTN-AI risk and baseline hypertension status and by quintile of HTN-AI score. The analysis 1300 includes a first graph 1302 and a second graph 1306. The first graph 1302 represents different combinations of HTN-AI risk and hypertension status, as indicated by a first legend 1304. The first legend 1304 shows a high HTN-AI/hypertension curve as a bolded solid line, a low HTN-AI/hypertension curve as a solid line, a high HTN-AI/no hypertension curve as a bolded dashed line, and a low HTN-AI/no hypertension curve as a dashed line. The second graph 1306 depicts five curves of different line types representing different percentiles (100th, 80th, 60th, 40th, and 20th) of HTN-AI score, as indicated by a second legend 1308. For both the first graph 1302 and the second graph 1306, the y-axis represents cumulative incidence, and the x-axis represents time in years. The curves in both graphs show an increasing trend over time, with higher risk categories and higher percentiles exhibiting steeper increases in the cumulative incidence of all-cause mortality.

FIG. 14 depicts an analysis 1400 of a cumulative incidence of heart failure, as stratified by HTN-AI risk and baseline hypertension status and by quintile of HTN-AI score. The analysis 1400 includes a first graph 1402 and a second graph 1406. The first graph 1402 represents different combinations of HTN-AI risk and hypertension status, as indicated by a first legend 1404. The first legend 1404 shows a high HTN-AI/hypertension curve as a bolded solid line, a low HTN-AI/hypertension curve as a solid line, a high HTN-AI/no hypertension curve as a bolded dashed line, and a low HTN-AI/no hypertension curve as a dashed line. The second graph 1406 depicts five curves of different line types representing different percentiles (100th, 80th, 60th, 40th, and 20th) of HTN-AI score, as indicated by a second legend 1408. For both the first graph 1402 and the second graph 1406, the y-axis represents cumulative incidence, and the x-axis represents time in years. The curves in both graphs show an increasing trend over time, with higher risk categories and higher percentiles exhibiting steeper increases in the cumulative incidence of heart failure.

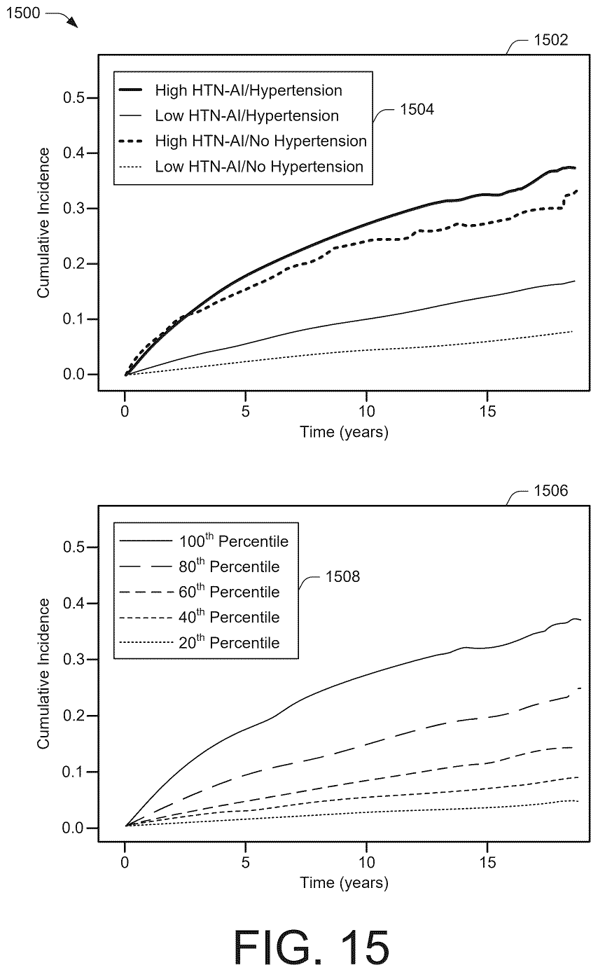

FIG. 15 depicts an analysis 1500 of a cumulative incidence of myocardial infarction, as stratified by HTN-AI risk and baseline hypertension status and by quintile of HTN-AI score. The analysis 1500 includes a first graph 1502 and a second graph 1506. The first graph 1502 represents different combinations of HTN-AI risk and hypertension status, as indicated by a first legend 1504. The first legend 1504 shows a high HTN-AI/hypertension curve as a bolded solid line, a low HTN-AI/hypertension curve as a solid line, a high HTN-AI/no hypertension curve as a bolded dashed line, and a low HTN-AI/no hypertension curve as a dashed line. The second graph 1506 depicts five curves of different line types representing different percentiles (100th, 80th, 60th, 40th, and 20th) of HTN-AI score, as indicated by a second legend 1508. For both the first graph 1502 and the second graph 1506, the y-axis represents cumulative incidence, and the x-axis represents time in years. The curves in both graphs show an increasing trend over time, with higher risk categories and higher percentiles exhibiting steeper increases in the cumulative incidence of myocardial infarction.

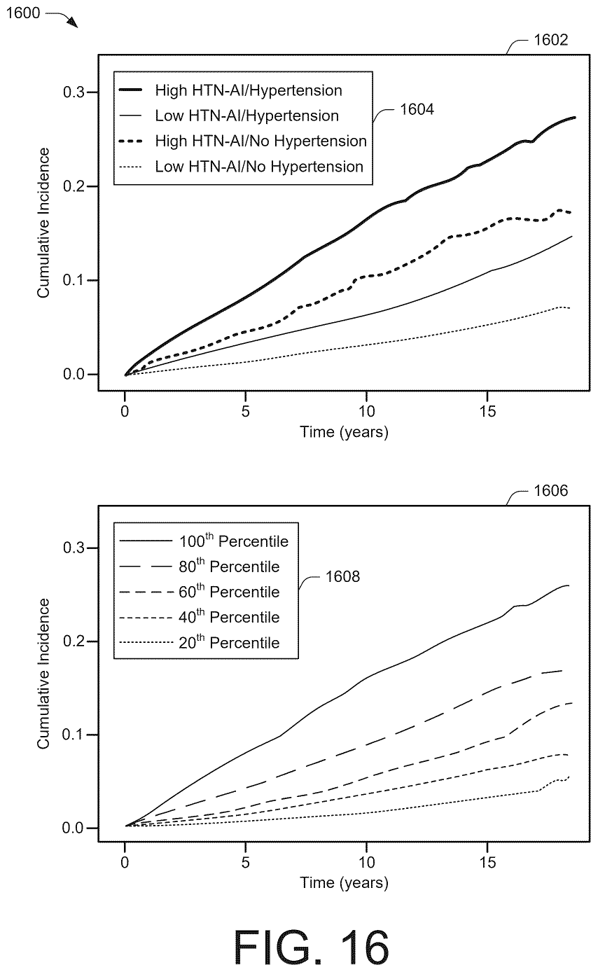

FIG. 16 depicts an analysis 1600 of a cumulative incidence of stroke, as stratified by HTN-AI risk and baseline hypertension status and by quintile of HTN-AI score. The analysis 1600 includes a first graph 1602 and a second graph 1606. The first graph 1602 represents different combinations of HTN-AI risk and hypertension status, as indicated by a first legend 1604. The first legend 1604 shows a high HTN-AI/hypertension curve as a bolded solid line, a low HTN-AI/hypertension curve as a solid line, a high HTN-AI/no hypertension curve as a bolded dashed line, and a low HTN-AI/no hypertension curve as a dashed line. The second graph 1606 depicts five curves of different line types representing different percentiles (100th, 80th, 60th, 40th, and 20th) of HTN-AI score, as indicated by a second legend 1608. For both the first graph 1602 and the second graph 1606, the y-axis represents cumulative incidence, and the x-axis represents time in years. The curves in both graphs show an increasing trend over time, with higher risk categories and higher percentiles exhibiting steeper increases in the cumulative incidence of stroke.

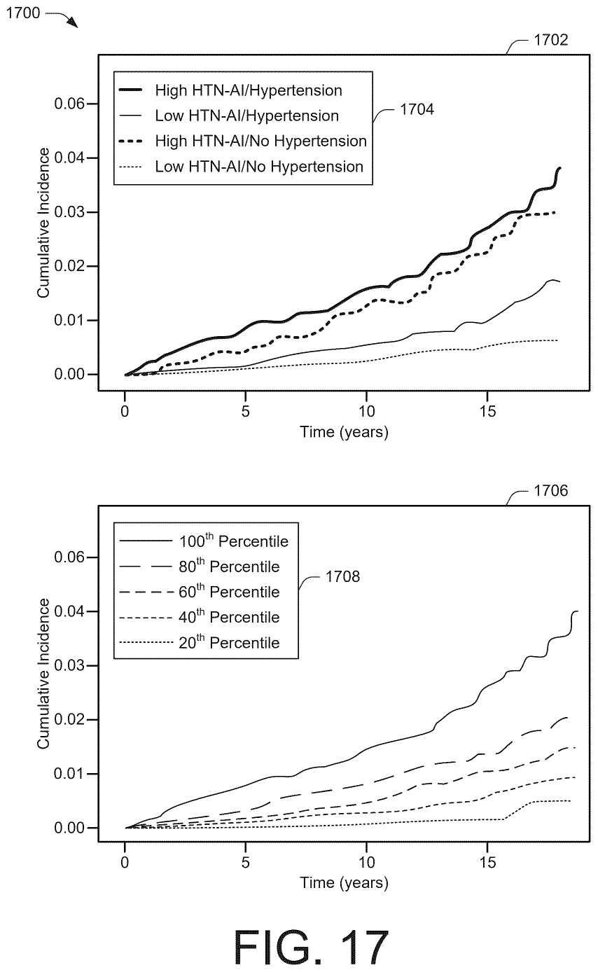

FIG. 17 depicts an analysis 1700 of a cumulative incidence of aortic dissection or rupture, as stratified by HTN-AI risk and baseline hypertension status and by quintile of HTN-AI score. The analysis 1700 includes a first graph 1702 and a second graph 1706. The first graph 1702 represents different combinations of HTN-AI risk and hypertension status, as indicated by a first legend 1704. The first legend 1704 shows a high HTN-AI/hypertension curve as a bolded solid line, a low HTN-AI/hypertension curve as a solid line, a high HTN-AI/no hypertension curve as a bolded dashed line, and a low HTN-AI/no hypertension curve as a dashed line. The second graph 1706 depicts five curves of different line types representing different percentiles (100th, 80th, 60th, 40th, and 20th) of HTN-AI score, as indicated by a second legend 1708. For both the first graph 1702 and the second graph 1706, the y-axis represents cumulative incidence, and the x-axis represents time in years. The curves in both graphs show an increasing trend over time, with higher risk categories and higher percentiles exhibiting steeper increases in the cumulative incidence of aortic dissection or rupture.

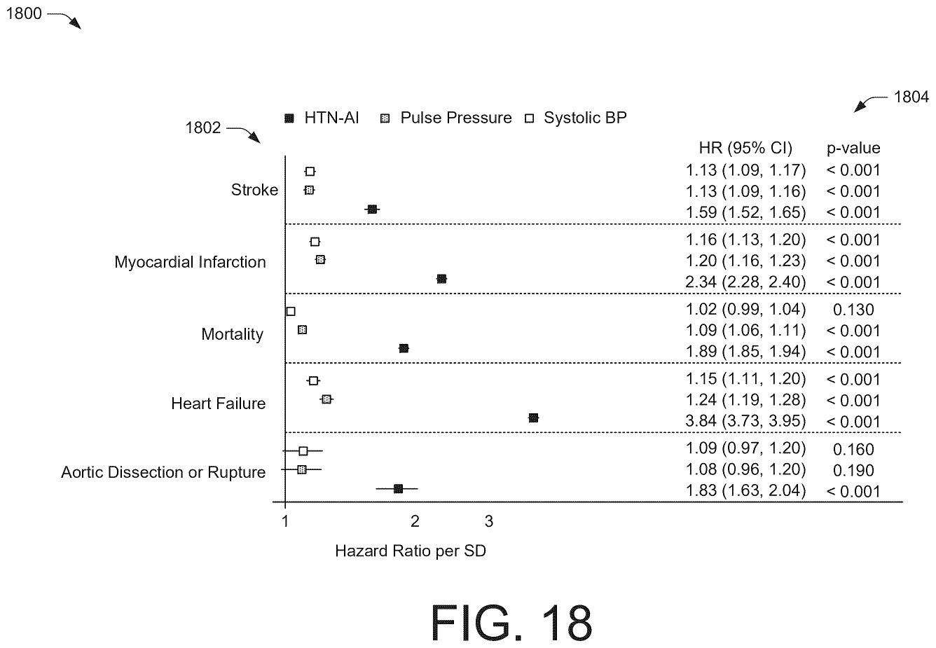

In regression models adjusted for age and sex, the HTN-AI score was significantly associated with mortality (HR per SD: 1.89 [1.85-1.94], p<0.001), HF (3.84 [3.73-3.95], p<0.001), MI (2.34 [2.28-2.40], p<0.001), stroke (1.59 [1.52-1.65], p<0.001), and aortic dissection or rupture (1.83 [1.63-2.04], p<0.001). FIG. 18 compares adjusted HRs per SD of HTN-AI score, SBP, and pulse pressure. Effect sizes were higher for HTN-AI than for SBP or pulse pressure across all outcomes. Findings were similar when comparing adjusted HRs for high HTN-AI risk to those for baseline hypertension diagnosis (FIG. 9).

FIG. 18 depicts an analysis 1800 comparing hazard ratios for various cardiovascular outcomes between HTN-AI and baseline blood pressure. The analysis 1800 includes a forest plot 1802 and statistics 1804. The forest plot 1802 displays hazard ratios per standard deviation (SD) and confidence intervals for different cardiovascular outcomes, including stroke, myocardial infarction, mortality, heart failure, and aortic dissection or rupture. Separate markers are shown for HTN-AI (black-filled squares), pulse pressure (shaded squares) and systolic blood pressure (BP) (white- or open-filled squares) for each outcome. The statistics 1804 provide numerical hazard ratio (HR) values with 95% confidence intervals and p-values for each outcome. The forest plot 1802 visually represents the relative strength of associations between HTN-AI, pulse pressure, and systolic BP with each cardiovascular outcome, while the statistics 1804 provide the precise numerical values for these associations.

### Model Interpretability

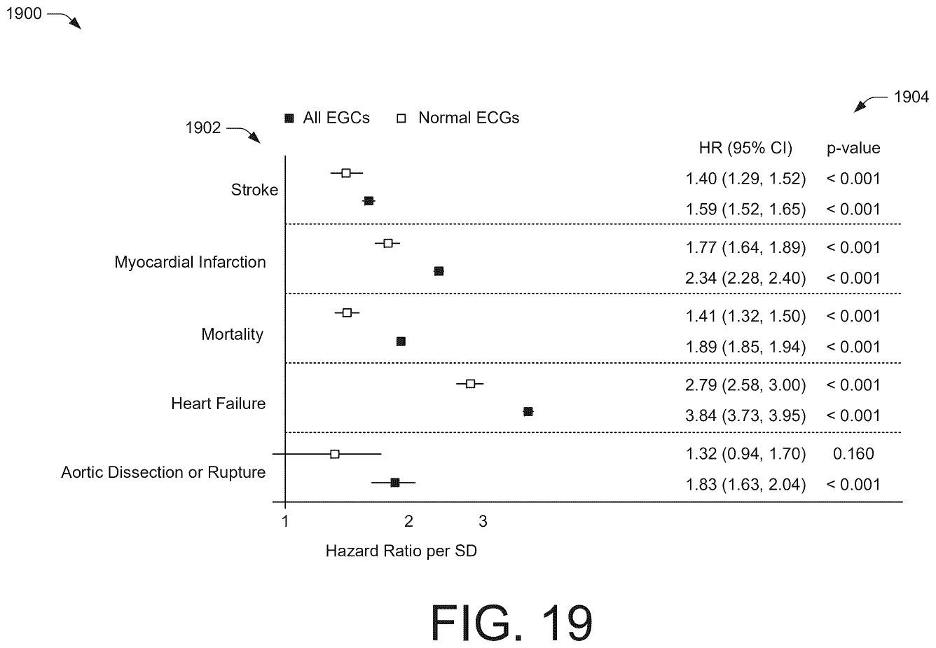

To assess whether associations between the HTN-AI score and CVD are sensitive to presence of ECG abnormalities, the associations were also examined in the subset of patients with ECGs interpreted by a cardiologist as normal. Among 17,096 patients in the BWH sample with normal ECGs, the age- and sex-adjusted association with the HTN-AI score remained significant for all outcomes except aortic dissection or rupture (p=0.16; p<0.001 for other outcomes; FIG. 19).

FIG. 19 depicts an analysis 1900 comparing hazard ratios for associations between HTN-AI score and various cardiovascular outcomes in patients with normal ECGs. The analysis 1900 includes a forest plot 1902 and statistics 1904. The forest plot 1902 displays hazard ratios per standard deviation (SD) and confidence intervals for different cardiovascular outcomes, including stroke, myocardial infarction, mortality, heart failure, and aortic dissection or rupture. Separate markers are shown for all ECGs (black-filled squares) and normal ECGs (white- or open-filled squares) for each outcome. The statistics 1904 provide numerical hazard ratio (HR) values with 95% confidence intervals and p-values for each outcome. The forest plot 1902 visually represents the relative strength of associations between HTN-AI score and cardiovascular outcomes for both all ECGs and normal ECGs, while the statistics 1904 provide the precise numerical values for these associations.

Saliency maps and median ECG waveforms were generated for 1024 randomly selected patients from the top and bottom deciles of HTN-AI score in the BWH sample, demonstrating that higher HTN-AI scores were associated with higher voltages and changes in QRS complex and T wave morphology.

### Discussion

HTN-AI, a deep learning model trained to detect hypertension using over 750,000 ECGs from over 100,000 patients, is presented. In an external sample of over 56,000 patients from another hospital, HTN-AI accurately identified hypertension, and high HTN-AI risk was associated with increased short-term incidence of hypertension diagnosis, which may be due to detection of subclinical ECG features associated with as yet undiagnosed hypertension. HTN-AI stratified risk of CVD regardless of baseline hypertension status, suggesting that it may be useful as a biomarker of hypertension-associated cardiovascular risk.

Office BP is often used to diagnose hypertension but does not always reflect ambulatory BP, leading to potential underdiagnosis of ambulatory hypertension. In contrast, HTN-AI detects electrocardiographic features of hypertension that result from the cumulative effect of high ambulatory BP on the myocardium. HTN-AI may therefore be useful to screen for ambulatory hypertension, even when office BP is normal, by identifying patients with subclinical ECG features of hypertension, which could prompt confirmatory testing with ambulatory BP monitoring.

Although HTN-AI was trained to identify hypertension from the ECG, the model also stratified the risk of hypertension-related CVD, regardless of baseline hypertension status. HTN-AI can therefore also provide a disease-specific biomarker for hypertension by detecting subtle electrocardiographic features of hypertension that are also associated with incident CVD. Greater normalized effect sizes for the HTN-AI score than for baseline BP across all CVD outcomes were observed, suggesting that HTN-AI is a better marker of cardiovascular risk than office BP measurements.

Given the size of the sample, which exceeded 150,000 patients, hypertension status was determined using office BPs and diagnosis codes rather than gold-standard ambulatory BP monitoring, creating potential for misclassification of hypertension status. However, misclassification was minimized by employing study cohorts that were purpose-built to limit data missingness and ascertainment bias, and the outcomes cohort was previously validated with respect to established cardiovascular risk models that included blood pressure measurements. Furthermore, the scale of the training sample allowed the model to identify electrocardiographic patterns associated with hypertension aggregated across 750,000 training ECGs, despite the potential misclassification of individual patients. As an example, HTN-AI predictions were more strongly associated with incident CVD than either baseline hypertension status or blood pressure, and high HTN-AI risk was associated with 3-fold odds of elevated 24-hour ambulatory BP, indicating that the model identified the physiological ECG signature of hypertension despite learning from EHR-derived training labels. Moreover, saliency maps and median waveforms suggest HTN-AI detects ECG features potentially reflective of hypertension-related changes, and the strong associations between HTN-AI predictions and CVD suggest that the model detects a biologically relevant signal in ECG waveforms. HTN-AI was also designed to learn features present in the ECG, but sequelae of hypertension are mediated in part by the effects of hypertension on other organs (e.g. the vasculature). However, the strong association of the HTN-AI output with non-myocardial diseases, such as aortic dissection and rupture, suggests that HTN-AI risk is a biomarker for hypertension itself rather than simply for its myocardial effects.

In this way, HTN-AI is a deep learning model that consistently discriminates prevalent hypertension from 12-lead ECGs and stratifies risk of incident mortality, HF, MI, stroke, and aortic dissection or rupture. This Example Application demonstrates the utilization of a deep learning model to facilitate the diagnosis of hypertension and serve as a novel digital biomarker for the risk of hypertension-associated cardiovascular disease.

### CONCLUSION

Although the invention has been described in language specific to structural features and/or methodological acts, it is to be understood that the invention defined in the appended claims is not necessarily limited to the specific features or acts described. Rather, the specific features and acts are disclosed as example forms of implementing the claimed invention.

What is claimed is:

1. A system for electrocardiogram-based hypertension prediction, comprising:
an electrocardiogram analysis module implemented in a non-transitory computer-readable storage medium, the electrocardiogram analysis module comprising:
a data preprocessor configured to normalize an electrocardiogram to generate a standardized input for the electrocardiogram-based hypertension prediction; and
a deep learning model including a neural network trained to identify features associated with hypertension from the standardized input and further including at least one dense layer trained to generate a hypertension risk prediction based on the identified features.

2. The system of claim 1, wherein the neural network comprises a convolutional neural network.

3. The system of claim 2, wherein the convolutional neural network comprises multiple convolutional blocks, each convolutional block including a plurality of convolutional layers, and wherein outputs of at least two convolutional layers of the plurality of convolutional layers are combined via a concatenation.

4. The system of claim 1, wherein the hypertension risk prediction comprises a hypertension probability score indicating a likelihood of hypertension or risk of developing hypertension.

5. The system of claim 4, wherein the hypertension risk prediction further comprises a hypertension risk stratification that categorizes individuals into risk groups based on the hypertension probability score compared to one or more thresholds.

6. The system of claim 1, wherein the at least one dense layer is further configured to generate auxiliary predictions based on the identified features.

7. The system of claim 6, wherein the auxiliary predictions comprise at least one of an age prediction, a sex prediction, or a medication prediction.

8. The system of claim 1, wherein the data preprocessor is further configured to upsample and zero-pad the electrocardiogram to generate the standardized input for the electrocardiogram-based hypertension prediction.

9. The system of claim 1, wherein the standardized input comprises a time-series of voltage measurements for each lead of the electrocardiogram that are sampled at a standardized frequency over a predetermined time period.

10. The system of claim 1, further comprising a training module configured to:
train the deep learning model using a training sample comprising a first portion of a model derivation subset of electrocardiogram training data, the electrocardiogram training data comprising electrocardiograms obtained from healthy patients and from patients diagnosed with hypertension; and
refine the trained deep learning model using a development sample comprising a second portion of the model derivation subset of the electrocardiogram training data.

11. The system of claim 10, wherein the training module is further configured to internally validate the trained and refined deep learning model using an internal test sample comprising a third portion of the model derivation subset of the electrocardiogram training data.

12. The system of claim 10, wherein the training module is further configured to externally validate the deep learning model using an external test subset of the electrocardiogram training data, wherein the model derivation subset and the external test subset comprise electrocardiogram recordings from different data sources.

13. A method for generating a hypertension risk prediction, said method comprising:
generating, by a data preprocessor, a standardized input for an electrocardiogram that is to be processed by a deep learning model trained to output the hypertension risk prediction;
extracting, by a neural network of the deep learning model, features of the standardized input;
outputting, by the neural network, an electrocardiogram feature map representing a high-dimensional abstraction of the features of the standardized input; and
generating, by an output layer of the deep learning model, the hypertension risk prediction based at least in part on the electrocardiogram feature map, wherein the hypertension risk prediction comprises at least a hypertension probability score.

14. The method of claim 13, wherein generating the standardized input comprises at least one of normalizing, upsampling, or zero-padding the electrocardiogram, and wherein the standardized input comprises a time-series of voltage measurements for each lead of the electrocardiogram that are sampled at a predetermined frequency over a predetermined time period.

15. The method of claim 13, wherein the neural network comprises a convolutional neural network having a plurality of convolutional blocks, each convolutional block of the plurality of convolutional blocks comprising at least one convolutional layer and at least one concatenation, and wherein the output layer comprises one or more dense layers.

16. The method of claim 13, further comprising, generating auxiliary predictions based on the electrocardiogram feature map, the auxiliary predictions comprising at least one of an age prediction, a sex prediction, or a medication prediction.

17. A method for hypertension prediction, comprising:
training a deep learning model to output a hypertension risk prediction, the training comprising:
initially training the deep learning model using a training sample of a model derivation subset of electrocardiogram training data by adjusting weights and biases of the deep learning model based on a difference between an output of the deep learning model for the hypertension risk prediction and a ground truth label; and
refining the initially trained deep learning model using a development sample of the model derivation subset of the electrocardiogram training data, the refining including adjusting hyperparameters of the deep learning model; and
generating the hypertension risk prediction for an individual using the trained deep learning model, the generating comprising:
generating, by a data preprocessor operatively connected to the trained deep learning model, a standardized input of an electrocardiogram obtained from the individual by preprocessing the electrocardiogram;
extracting, by the trained deep learning model, features of the standardized input;
generating, by the trained deep learning model, an electrocardiogram feature map summarizing the features of the standardized input; and
outputting, by the trained deep learning model, the hypertension risk prediction based on the electrocardiogram feature map.

18. The method of claim 17, wherein the training further comprises:
internally validating the refined deep learning model using an internal test sample of the model derivation subset of the electrocardiogram training data; and
externally validating the internally validated deep learning model using an external test subset of the electrocardiogram training data.

19. The method of claim 17, wherein the hypertension risk prediction comprises a hypertension probability score that indicates a likelihood of the individual having hypertension and a hypertension risk stratification that categorizes the individual into risk groups based on the hypertension probability score compared to one or more thresholds.

20. The method of claim 17, further comprising, generating auxiliary predictions based on the electrocardiogram feature map, the auxiliary predictions comprising at least one of an age prediction, a sex prediction, or a medication prediction.
